SEQUENCE LISTING (113 sequences)

SEQ ID NO: 1 - RNA, 22 nt, Homo sapiens
  misc_RNA (1)..(22): SEQ ID NO 1 is hsa-miR-211, an endogenous microRNA in humans.
uucccuuugucauccuucgccu

SEQ ID NO: 2 - RNA, 22 nt, Homo sapiens
  misc_RNA (1)..(22): SEQ ID NO 2 is hsa-miR 34b, an endogenous microRNA in humans.
caaucacuaacuccacugccau

SEQ ID NO: 3 - RNA, 22 nt, Homo sapiens
  misc_RNA (1)..(22): SEQ ID NO 3 is hsa-miR-375, an endogenous microRNA in humans.
uuuguucguucggcucgcguga

SEQ ID NO: 4 - RNA, 22 nt, Homo sapiens
  misc_RNA (1)..(22): SEQ ID NO 4 is hsa-miR-204, an endogenous microRNA in humans.
uucccuuugucauccuaugccu

SEQ ID NO: 5 - RNA, 22 nt, Homo sapiens
  misc_RNA (1)..(22): SEQ ID NO 5 is hsa-miR-99a, an endogenous microRNA in humans.
aacccguagauccgaucuugug

SEQ ID NO: 6 - RNA, 22 nt, Homo sapiens
  misc_RNA (1)..(22): SEQ ID NO 6 is hsa-miR-16, an endogenous microRNA in humans.
uagcagcacguaaauauuggcg

SEQ ID NO: 7 - RNA, 22 nt, Homo sapiens
  misc_feature (1)..(22): SEQ ID NO 7 is hsa-miR-let-7a, an endogenous microRNA in humans.
ugagguaguagguuguauaguu

SEQ ID NO: 8 - RNA, 22 nt, Homo sapiens
  misc_RNA (1)..(22): SEQ ID NO 8 is hsa-miR-let-7c, an endogenous microRNA in humans.
ugagguaguagguuguaugguu

SEQ ID NO: 9 - RNA, 22 nt, Homo sapiens
  misc_RNA (1)..(22): SEQ ID NO 9 is hsa-miR-let-7d, an endogenous microRNA in humans.
agagguaguagguugcauaguu

SEQ ID NO: 10 - RNA, 22 nt, Homo sapiens
  misc_RNA (1)..(22): SEQ ID NO 10 is hsa-miR-let-7e, an endogenous microRNA in humans.
ugagguaggagguuguauaguu

SEQ ID NO: 11 - RNA, 22 nt, Homo sapiens
  misc_RNA (1)..(22): SEQ ID NO 11 is hsa-miR-let-7f, an endogenous microRNA in humans.
ugagguaguagauuguauaguu

SEQ ID NO: 12 - RNA, 22 nt, Homo sapiens
  misc_RNA (1)..(22): SEQ ID NO 12 is hsa-miR-let-7g, an endogenous microRNA in humans.
ugagguaguaguuuguacaguu

SEQ ID NO: 13 - RNA, 22 nt, Homo sapiens
  misc_RNA (1)..(22): SEQ ID NO 13 is hsa-miR-let-7i, an endogenous microRNA in humans.
ugagguaguaguuugugcuguu

SEQ ID NO: 14 - RNA, 24 nt, Homo sapiens
  misc_RNA (1)..(24): SEQ ID NO 14 is hsa-miR-125a, an endogenous microRNA in humans.
ucccugagacccuuuaaccuguga

SEQ ID NO: 15 - RNA, 22 nt, Homo sapiens
  misc_RNA (1)..(22): SEQ ID NO 15 is hsa-miR-125b, an endogenous microRNA in humans.
ucccugagacccuaacuuguga

SEQ ID NO: 16 - RNA, 22 nt, Homo sapiens
  misc_RNA (1)..(22): SEQ ID NO 16 is hsa-miR-15b, an endogenous microRNA in humans.
uagcagcacaucaugguuuaca

SEQ ID NO: 17 - RNA, 23 nt, Homo sapiens
  misc_RNA (1)..(23): SEQ ID NO 17 is hsa-miR-199a, an endogenous microRNA in humans.
cccaguguucagacuaccuguuc

SEQ ID NO: 18 - RNA, 22 nt, Homo sapiens
  misc_RNA (1)..(22): SEQ ID NO 18 is hsa-miR-21, an endogenous microRNA in humans.
uagcuuaucagacugauguuga

SEQ ID NO: 19 - RNA, 22 nt, Homo sapiens
  misc_RNA (1)..(22): SEQ ID NO 19 is hsa-miR-214, an endogenous microRNA in humans.
acagcaggcacagacaggcagu

SEQ ID NO: 20 - RNA, 23 nt, Homo sapiens
  misc_RNA (1)..(23): SEQ ID NO 20 is hsa-miR-221, an endogenous microRNA in humans.
agcuacauugucugcuggguuuc

SEQ ID NO: 21 - RNA, 21 nt, Homo sapiens
  misc_RNA (1)..(21): SEQ ID NO 21 is hsa-miR-222, an endogenous microRNA in humans.
agcuacaucuggcuacugggu

SEQ ID NO: 22 - RNA, 21 nt, Homo sapiens
  misc_RNA (1)..(21): SEQ ID NO 22 is hsa-miR-23a, an endogenous microRNA in humans.
aucacauugccagggauuucc

SEQ ID NO: 23 - RNA, 21 nt, Homo sapiens
  misc_RNA (1)..(21): SEQ ID NO 23 is hsa-miR-23b, an endogenous microRNA in humans.
aucacauugccagggauuacc

SEQ ID NO: 24 - RNA, 22 nt, Homo sapiens
  misc_RNA (1)..(22): SEQ ID NO 24 is hsa-miR-26a, an endogenous microRNA in humans.
ccuauucuugguuacuugcacg

SEQ ID NO: 25 - RNA, 23 nt, Homo sapiens
  misc_RNA (1)..(23): SEQ ID NO 25 is hsa-miR-30c, an endogenous microRNA in humans.
uguaaacauccuacacucucagc

SEQ ID NO: 26 - RNA, 22 nt, Homo sapiens
  misc_RNA (1)..(22): SEQ ID NO 26 is hsa-miR-320, an endogenous microRNA in humans.
aaaagcuggguugagagggcga

SEQ ID NO: 27 - RNA, 22 nt, Homo sapiens
  misc_RNA (1)..(22): SEQ ID NO 27 is hsa-miR-let-7b, an endogenous microRNA in humans.
ugagguaguagguugugugguu

SEQ ID NO: 28 - DNA, 60 bp, Homo sapiens
  misc_signal (1)..(60): Genomic DNA located on chr990255724-90255784, which codes for an ncRNA.
cttaatgtatctaaggttggcttaatcctgtctaaaatcaaggtattggactagaacaat

SEQ ID NO: 29 - DNA, 60 bp, Homo sapiens
  misc_signal (1)..(60): Genomic DNA located on chr1278691877-78691937, which encodes for an ncRNA.
ttaaatacatgtttctaatattttagtgttttgtatttaggttatttattctttgtatct

SEQ ID NO: 30 - DNA, 60 bp, Homo sapiens
  misc_signal (1)..(60): Genomic DNA located on human chr5141677414-141677474, which encodes for an ncRNA.
attccaaggcctattaaaatttctgagcattgcccatttcttttgctttatctgtaggac

SEQ ID NO: 31 - DNA, 60 bp, Homo sapiens
  misc_signal (1)..(60): Genomic DNA located on human chr1683258512-83258572, which encodes for an ncRNA.
ttgcttttgtatttgtaattttatgacatttcgaagtttctgtgtcttaactctttttaa

SEQ ID NO: 32 - DNA, 60 bp, Homo sapiens
  misc_signal (1)..(60): Genomic DNA located on human chr205121228-5121288, which encodes for an ncRNA.
attcaaatcttcatgaatggcatcacttgctttagacccattttttttttattggtcaga

SEQ ID NO: 33 - DNA, 60 bp, Homo sapiens
  misc_signal (1)..(60): Genomic DNA located on human chr1579084787-79084847, which encodes an ncRNA.
tattgaatgtacacaagtaatgatggagtttgattatatggttcattttcatttatccta

SEQ ID NO: 34 - DNA, 60 bp, Homo sapiens
  misc_signal (1)..(60): Genomic DNA located on human chr371578153-71578213, which encodes an ncRNA.
ttttctctgcttattaccctccctgtttcctcctgggctgccaacaacatattatattac

SEQ ID NO: 35 - DNA, 60 bp, Homo sapiens
  misc_signal (1)..(60): Genomic DNA located on human chr4147079236-147079296, which codes for an ncRNA.
gtttccattgaagaatattacgctcggggacaagccaggtgcacgaccaaaaatttaaaa

SEQ ID NO: 36 - DNA, 60 bp, Homo sapiens
  misc_signal (1)..(60): Genomic DNA located on human chr1046553108-46553168, which encodes an ncRNA.
tatttgtgactaactgtataacaggttattttagtttctgttctgtgggaagtgtaaagc

SEQ ID NO: 37 - DNA, 60 bp, Homo sapiens
  misc_signal (1)..(60): Genomic DNA located on human chr2037023983-37024043, which encodes an ncRNA.
acgcactgggcgttgaagcagtgctttctggattaaatacgaaatactgatgtcacaagc

SEQ ID NO: 38 - DNA, 60 bp, Homo sapiens
  misc_signal (1)..(60): Genomic DNA located on human chr117105017-17105083, which encodes an ncRNA.
gtaaacaaggtctcacaaaaacagctgaccacaccataaaatcactgagaccgagcctgc

SEQ ID NO: 39 - DNA, 60 bp, Homo sapiens
  misc_signal (1)..(60): Genomic DNA located on human chr59033546-9033606, which encodes an ncRNA.
tgatcctgtaaatcatgtttttgtttattccccaacactaacctcactcctttactattc

SEQ ID NO: 40 - DNA, 60 bp, Homo sapiens
  misc_signal (1)..(60): Genomic DNA located on human chr973731470-73731530, which encodes an ncRNA.
gttaaagactaaattagtgatttgggagatcaagccaatgggcttgtctaaaacctagtc

SEQ ID NO: 41 - DNA, 60 bp, Homo sapiens
  misc_signal (1)..(60): Genomic DNA located on human chr9110821312-110821372, which encodes an ncRNA.
ctcgtaacactgggaaaactgcatacaatatttagaaggaacactaatacagcagaatct

SEQ ID NO: 42 - DNA, 60 bp, Homo sapiens
  misc_signal (1)..(60): Genomic DNA located on human chr7127930199-127930259, which encodes an ncRNA
tagacataggttagagggggaacagtgcttaaaattccatggaactaagattttttttat

SEQ ID NO: 43 - DNA, 60 bp, Homo sapiens
  misc_signal (1)..(60): Genomic DNA located on human chr1149822378-149822438, which encodes an ncRNA
cttaatccacatttgttttgaatttttctgattcctatagtaccatactctgatgcctat

SEQ ID NO: 44 - DNA, 60 bp, Homo sapiens
  misc_signal (1)..(60): Genomic DNA located on human chr11125333602-125333662, which encodes an ncRNA
agccaactcacgcttagggagaactcataccgcctaaatataaaggcaattatatatata

SEQ ID NO: 45 - DNA, 60 bp, Homo sapiens
  misc_signal (1)..(60): Genomic DNA located on human chr988742481-88742541, which encodes an ncRNA
atttagagaacatttaaatctgtaaggaagaaaataataacgactcacaattacatcatt

SEQ ID NO: 46 - DNA, 60 bp, Homo sapiens
  misc_signal (1)..(60): Genomic DNA located on human chr162929717-62929777, which encodes an ncRNA
ctattggagtgcatataaattcttgatagcaccagttctcaaatatgaggtgttaaaaac

SEQ ID NO: 47 - DNA, 60 bp, Homo sapiens
  misc_signal (1)..(60): Genomic DNA located on human chr214697555-14697615, which encodes an ncRNA
gtatttgtaatatttgatttgcttttagcagagaaaacaataaaagaatccaggaaaagt

SEQ ID NO: 48 - DNA, 60 bp, Homo sapiens
  misc_signal (1)..(60): Genomic DNA located on human chr12108979299-108979359, which encodes an ncRNA
acttatattcagagcatgtggattgggactctgaagaacaactttatggtggacaccttc

SEQ ID NO: 49 - DNA, 60 bp, Homo sapiens
  misc_signal (1)..(60): Genomic DNA located on human chr1723889984-23890044, which encodes an ncRNA
cgatggcttgtcattgtcccctgtcctggaacaataaagcaagtgccttgtggtctcact

SEQ ID NO: 50 - DNA, 60 bp, Homo sapiens
  misc_signal (1)..(60): Genomic DNA located on human chr1777136100-77136160, which encodes an ncRNA
gttattataaccaatgcagactttaaaatcccacctggacatcgggtgagaggaggaggg

SEQ ID NO: 51 - DNA, 60 bp, Homo sapiens
  misc_signal (1)..(60): Genomic DNA located on human chr2241669818-41669878, which encodes an ncRNA
atgtattttaggtaataagatttttatcaattgtgtctttttctttctttgctttctata

SEQ ID NO: 52 - DNA, 60 bp, Homo sapiens
  misc_signal (1)..(60): Genomic DNA located on human chr2153150591-153150651, which encodes an ncRNA
ttttcttagtctttttactttttccattatattctttgatatatttgagaaaatggcttg

SEQ ID NO: 53 - DNA, 60 bp, Homo sapiens
  misc_signal (1)..(60): Genomic DNA located on human chr271136422-71136482, which encodes an ncRNA
atatatttagatagtactatgtttcgttttcttccgatttcagggatccttttgtcttac

SEQ ID NO: 54 - DNA, 60 bp, Homo sapiens
  misc_signal (1)..(60): Genomic DNA located on human chr1531185251-31185311, which encodes an ncRNA
ttttttttcttgtcctattacaacattttataaccttatgaatcaatggatacttttatt

SEQ ID NO: 55 - DNA, 60 bp, Homo sapiens
  misc_signal (1)..(60): Genomic DNA located on human chr162174567-62174627, which encodes an ncRNA
cctctccccttcttgcaaaaaatattctatattcaagtttaggagaaaacagaatacttg

SEQ ID NO: 56 - DNA, 60 bp, Homo sapiens
  misc_signal (1)..(60): Genomic DNA located on human chr1372871205-72871265, which encodes an ncRNA
aaaaaatgaggcaatctcatcccgaaactatttaaaactactctcctctctcttgataaa

SEQ ID NO: 57 - DNA, 60 bp, Homo sapiens
  misc_signal (1)..(60): Genomic DNA located on human chr6130070887-130070947, which encodes an ncRNA
ttgttgttttgggagataccctgtttagcctgaaaggattgagatactgtaattgctatg

SEQ ID NO: 58 - DNA, 60 bp, Homo sapiens
  misc_signal (1)..(60): Genomic DNA located on human chr624809227-24809287, which encodes an ncRNA
atttatttgactctaaaatgacaatataacaactataaggaattgatatgatgtccttac

SEQ ID NO: 59 - DNA, 60 bp, Homo sapiens
  misc_signal (1)..(60): Genomic DNA located on human chr2056684930-56684990, which encodes an ncRNA
tgtgcagcagtatcaaaggtccttaaattctcaacaatgaaggaaaaacaaaaacccatt

SEQ ID NO: 60 - DNA, 60 bp, Homo sapiens
  misc_signal (1)..(60): Genomic DNA located on human chr1753763585-53763645, which encodes an ncRNA
actaacagcactggagggtgtagtgtttcctactttatggatgagtgtactgtgggcttc

SEQ ID NO: 61 - DNA, 60 bp, Homo sapiens
  misc_signal (1)..(60): Genomic DNA located on human chr135973194-35973254, which encodes an ncRNA
catacattgccctcactaatcaatggacatttcagcatttcattactaattttgagagaa

SEQ ID NO: 62 - DNA, 60 bp, Homo sapiens
  misc_signal (1)..(60): Genomic DNA located on human chrX74877202-74877262, which encodes an ncRNA
aatcgttaatatcattgatattaagtaatttccccactctgagtgaatactttgatgatg

SEQ ID NO: 63 - DNA, 60 bp, Homo sapiens
  misc_signal (1)..(60): Genomic DNA located on human chr1963554003-63554063, which encodes an ncRNA
ctcacacgcaccctttcactgtttcttagtaatttgtttgtctacagacacagacgggaa

SEQ ID NO: 64 - DNA, 60 bp, Homo sapiens
  misc_signal (1)..(60): Genomic DNA located on human chr1916624313-16624373, which encodes an ncRNA
ttgcctaggaacaaagaaggcttgtgaggagatcaaggaacttacatgaaattgtaggaa

SEQ ID NO: 65 - DNA, 60 bp, Homo sapiens
  misc_signal (1)..(60): Genomic DNA located on human chr5133735401-133735461, which encodes an ncRNA
caccgttttactttttgtggggaggggaagcagtatcaaaatttcttcgggcgccagaac

SEQ ID NO: 66 - DNA, 60 bp, Homo sapiens
  misc_signal (1)..(60): Genomic DNA located on human chr1532932757-32932817, which encodes an ncRNA
tcctttttatcttgcactcagatggtatttttagagatgtttcttcaatcaaaacaagat

SEQ ID NO: 67 - DNA, 60 bp, Homo sapiens
  misc_signal (1)..(60): Genomic DNA located on human chr2056644135-56644195, which encodes an ncRNA
ttccctattccatcctttgatgggtggcctgttacacgtttggaaataaaaacaagtggg

SEQ ID NO: 68 - DNA, 60 bp, Homo sapiens
  misc_signal (1)..(60): Genomic DNA located on human chr128885069-28885129, which encodes an ncRNA
caaaaagaaatcttgtttacttgtaaaaatatagactacctcctacttgccaccgttaag

SEQ ID NO: 69 - DNA, 60 bp, Homo sapiens
  misc_signal (1)..(60): Genomic DNA located on human chr1143921603-43921663, which encodes an ncRNA
agagttcgcactgggaagagttaaaaaataaacatttacaaggacgaggaaagcggcccc

SEQ ID NO: 70 - DNA, 60 bp, Homo sapiens
  misc_signal (1)..(60): Genomic DNA located on human chr1199447658-199447718, which encodes an ncRNA
agttatagaggaggctcaggaggatctggggaaacgggaccagagggtaagatgggttat

SEQ ID NO: 71 - DNA, 60 bp, Homo sapiens
  misc_signal (1)..(60): Genomic DNA located on human chrX152518379-152518439, which encodes an ncRNA
aaaattccaagagggccttttcgaatagacgcatgaacggtgtttgtgtctggatcgtcc

SEQ ID NO: 72 - DNA, 60 bp, Homo sapiens
  misc_signal (1)..(60): Genomic DNA located on human chr1017319132-17319192, which encodes an ncRNA
aattacatatatttagcaggatttttatttgccgtgatatataggataatttagtctttg

SEQ ID NO: 73 - DNA, 60 bp, Homo sapiens
  misc_signal (1)..(60): Genomic DNA located on human chr2042809173-42809233, which encodes an ncRNA
aaagtttccccataatgctcttgcaagtgaatatgagctgtggtttttgcagtggagtta

SEQ ID NO: 74 - DNA, 60 bp, Homo sapiens
  misc_signal (1)..(60): Genomic DNA located on human chr1320082609-20082669, which encodes an ncRNA
tatattacaggagttgtctatacgattatagtacctgattctttggacatatcattgaac

SEQ ID NO: 75 - DNA, 60 bp, Homo sapiens
  misc_signal (1)..(60): Genomic DNA located on human chr12111954-112014, which encodes an ncRNA
caagtgggaagaaaaagagccctacgtggtgacattctggaaggatatcatggatgattg

SEQ ID NO: 76 - DNA, 60 bp, Homo sapiens
  misc_signal (1)..(60): Genomic DNA located on human chr9123585590-123585650, which encodes an ncRNA
tgtttctaatgcatactgtgaaaggaaaaaaaatcacagttatccaccttctcagggcct

SEQ ID NO: 77 - DNA, 60 bp, Homo sapiens
  misc_signal (1)..(60): Genomic DNA located on human chr2237591429-37591489, which encodes an ncRNA
aagtttccacggcaggttctcgtagcacctttggtcaagaactgaaggggcctttagatg

SEQ ID NO: 78 - DNA, 60 bp, Homo sapiens
  misc_signal (1)..(60): Genomic DNA located on human chr72727349-2727409, which encodes an ncRNA
atggaaaggccaggggagggtggagctcaggagtggaagcacagggactaaggcctgagc

SEQ ID NO: 79 - DNA, 60 bp, Homo sapiens
  misc_signal (1)..(60): Genomic DNA located on human chr65748566-5748626, which encodes an ncRNA
gtgtgtactgatgtcaaagaccaaatggatgcccctgtggccaacagtaccttgttaaac

SEQ ID NO: 80 - DNA, 60 bp, Homo sapiens
  misc_signal (1)..(60): Genomic DNA located on human chr634135488-34135548, which encodes an ncRNA
cacacagcccagtctgcctgtgatggtaatataaatcaaaccagggacccaagagcccag

SEQ ID NO: 81 - DNA, 60 bp, Homo sapiens
  misc_signal (1)..(60): Genomic DNA located on human chr1162137113-62137173, which encodes an ncRNA
ctaggggcggagtttctcgtaaggggcaaggccaaggcatcttgtattggggctgacagg

SEQ ID NO: 82 - DNA, 60 bp, Homo sapiens
  misc_signal (1)..(60): Genomic DNA located on human chr8102702286-102702346, which encodes an ncRNA
gaagtcagaaactaaagttcccttctaagacaaaatcctgctttggagaaaaaaaaaaat

SEQ ID NO: 83 - DNA, 60 bp, Homo sapiens
  misc_signal (1)..(60): Genomic DNA located on human chr2118447564-118447624, which encodes an ncRNA
tcagtttgatgattgggctagggaatgaagctttgccacttaggagataaaccccataac

SEQ ID NO: 84 - DNA, 60 bp, Homo sapiens
  misc_signal (1)..(60): Genomic DNA located on human chr738356521-38356581, which encodes an ncRNA
ttattaaaaggactgaggatcatgtgtgttggtgtgtcccggagtctgctctaaggtaaa

SEQ ID NO: 85 - DNA, 60 bp, Homo sapiens
  misc_signal (1)..(60): Genomic DNA located on human chr1422064398-22064459, which encodes an ncRNA
tgaggggagaggggatgggaaacattagggctgggttcatgtaaagggaccagcattgtg

SEQ ID NO: 86 - DNA, 60 bp, Homo sapiens
  misc_signal (1)..(60): Genomic DNA located on human chr894066049-94066109, which encodes an ncRNA
aaaattattttatcttaagtttccatttagttgattaaaaatagggttatgaatgttaaa

SEQ ID NO: 87 - DNA, 60 bp, Homo sapiens
  misc_signal (1)..(60): Genomic DNA located on human chr2061945425-61945485, which encodes an ncRNA
gggatatttttaaggggaaggggaaggatagagagaaagtgcaggcagggctggggtctg

SEQ ID NO: 88 - DNA, 60 bp, Homo sapiens
  misc_signal (1)..(60): Genomic DNA located on human chr4191143103-191143163, which encodes an ncRNA
ctgttgcttattctgggagcccaatcctaggctgagggaggtgggcgcggggctgtttaa

SEQ ID NO: 89 - DNA, 60 bp, Homo sapiens
  misc_signal (1)..(60): Genomic DNA located on human chr2218347912-18347972, which encodes an ncRNA
ggacacagagagtcatgcaccagcccagtgagaaccaattcagagccaaatgccaaaccc

SEQ ID NO: 90 - DNA, 22 bp, Artificial Sequence
  forward primer
ttccctttgtcatccttcgcct

SEQ ID NO: 91 - DNA, 22 bp, Artificial Sequence
  reverse primer
aggcgaaggatgacaaagggaa

SEQ ID NO: 92 - DNA, 19 bp, Artificial Sequence
  forward primer
ctcctccaccccagcctgc

SEQ ID NO: 93 - DNA, 23 bp, Artificial Sequence
  reverse primer
tgagtgaaggatgaggcccacag

SEQ ID NO: 94 - DNA, 28 bp, Artificial Sequence
  forward primer
cagaagaataggtggtatgttcctaatg

SEQ ID NO: 95 - DNA, 27 bp, Artificial Sequence
  reverse primer
gcagcatctgtaagattcttctatctg

SEQ ID NO: 96 - DNA, 28 bp, Artificial Sequence
  forward primer
taacatttgtaaagggaaaattagcact

SEQ ID NO: 97 - DNA, 28 bp, Artificial Sequence
  reverse primer
agtgctaattttccctttacaaatgtta

SEQ ID NO: 98 - DNA, 28 bp, Artificial Sequence
  forward primer
ttactgacaaaaagggaaaatacgaaac

SEQ ID NO: 99 - DNA, 28 bp, Artificial Sequence
  reverse primer
gtttcgtattttccctttttgtcagtaa

SEQ ID NO: 100 - DNA, 24 bp, Artificial Sequence
  forward primer
gcaagcagtgtgtcgcttctgcac

SEQ ID NO: 101 - DNA, 23 bp, Artificial Sequence
  reverse primer
gcaagaggataggagagggcaac

SEQ ID NO: 102 - RNA, 24 nt, Homo sapiens
  misc_RNA (1)..(24): miR-67
ucacaaccuccuagaaagaguaga

SEQ ID NO: 103 - DNA, 57 bp, Artificial Sequence
  probe for ncRNA array
attccaaggcctattaaaatttctgagcattgcccatttcttttgctttatctgtag

SEQ ID NO: 104 - DNA, 60 bp, Artificial Sequence; identical to SEQ ID NO: 35
  probe for ncRNA array

SEQ ID NO: 105 - DNA, 60 bp, Artificial Sequence
  probe for ncRNA array
ggttctatgtccctgcggctatgtttccagtgtcctctgggtgtttccaagagcaacagg

SEQ ID NO: 106 - DNA, 60 bp, Artificial Sequence
  probe for ncRNA array
cagtccctcaccagtcgccagcctttccaaggtgagcttcagcattgaggccaaggtgcg

SEQ ID NO: 107 - DNA, 60 bp, Artificial Sequence; identical to SEQ ID NO: 29
  primer for ncRNA array

SEQ ID NO: 108 - DNA, 60 bp, Artificial Sequence; identical to SEQ ID NO: 37
  probe for ncRNA array

SEQ ID NO: 109 - DNA, 60 bp, Artificial Sequence; identical to SEQ ID NO: 32
  probe for ncRNA array

SEQ ID NO: 110 - DNA, 60 bp, Artificial Sequence; identical to SEQ ID NO: 33
  probe for ncRNA array

SEQ ID NO: 111 - DNA, 60 bp, Artificial Sequence; identical to SEQ ID NO: 28
  probe for ncRNA array

SEQ ID NO: 112 - DNA, 60 bp, Artificial Sequence; identical to SEQ ID NO: 35
  probe for ncRNA array

SEQ ID NO: 113 - DNA, 60 bp, Artificial Sequence; identical to SEQ ID NO: 30
  probe for ncRNA array

(19) United States
(12) Patent Application Publication
(10) Pub. No.: US 2011/0143948 A1
(43) Pub. Date: Jun. 16, 2011

(54) MOLECULAR SIGNATURES AND BIOMARKERS ASSOCIATED WITH MELANOMA AND METHODS OF USE THEREOF

(76) Inventor: Ranjan Perera, Savannah, GA (US)

(21) Appl. No.: 12/865,456

(22) PCT Filed: Jan. 30, 2009
(86) PCT No.: PCT/US09/32511

§ 371 Date: Dec. 2, 2010

Related U.S. Application Data
(60) Provisional application No. 61/063,232, filed on Feb. 1, 2008.
(60) Provisional application No. 61/063,651, filed on Feb. 4, 2008.

(51) Int. Cl. C40B 30/00; C12Q 1/68; G01N 33/53

(52) U.S. Cl. 506/7; 435/6.12; 435/6.14; 435/6.11; 436/94

(57) ABSTRACT

Described herein are methods for evaluating the risk of melanoma in subjects. The methods involve detecting and quantifying one or more biomarkers associated with melanoma in a biological sample from a subject.

### CROSS REFERENCE TO RELATED APPLICATION

This application claims priority upon U.S. Provisional Application Ser. No. 61/063,232 filed on Feb. 1, 2008, and to U.S. Provisional Application Ser. No. 61/063,651 filed on Feb. 4, 2008. These applications are hereby incorporated by reference in its entireties for all of their teachings.

### CROSS REFERENCE TO SEQUENCE LISTING

ncRNAs, including miRNAs, and genomic DNA encoding ncRNA described herein are referred to by a sequence identifier number (SEQ ID NO). The SEQ ID NO corresponds numerically to the sequence identifiers <400>1, <400>2, etc. The Sequence Listing, in written computer readable format (CFR), is incorporated by reference in its entirety.

### BACKGROUND

Malignant melanoma is the most lethal form of skin cancer in the United States. Typically abnormal cell division of melanocytes and of other ill-defined skin cell types cause this malady. Multiple clinical subtypes of melanomas exist such as acral lentiginious melanoma (ALM) which accounts for about 50% of melanoma in non-Caucasian populations and superficial spreading melanoma (SSM). In 2006 alone, newly reported cases of melanoma totaled 62,190, and in that same year, melanoma resulted in approximately 8,000 deaths.

This malady widely afflicts multiple races, genders, and ethnicities, and the molecular causes for this disease are poorly understood. It is theorized that UV exposure from the sun causes skin cancers such as melanoma, and in general, much research has focused on the effects of UV radiation damage and abnormal repair of DNA within these diseased cells. In particular, research has focused on mutated or deficient DNA proofreading and repair pathways, which include nucleotide excision repair pathways, the translesion synthesis repair pathway, and mismatch repair pathways. It has been further theorized that these mutated pathways, may lead to gross chromosomal rearrangements, aberrant cell signaling, or a multitude of other abnormal cellular activities. To date, however, little if any focus has attributed non-coding RNA (ncRNA), including microRNA (miRNA), involvement in melanoma or aberrant epigenetic regulation of these ncRNAs in melanoma development and progression.

MicroRNAs are small, non-coding RNAs with an average length of about twenty-one to twenty-three base pairs. Though hundreds of miRNAs have been discovered in a variety of organisms, little is known about their cellular function. They have been implicated, for example, in post-transcriptional regulation, regulation of developmental timing and pattern formation, restriction of differentiation potential, regulation of insulin secretion, resistance to viral infection, and in genomic rearrangements associated with carcinogenesis and other genetic disorders, such as fragile X syndrome. While miRNAs have been linked to post-transcriptional and developmental regulations, other non-coding RNAs have little or no known function.

Recent evidence suggests that the number of unique miRNAs in humans alone could exceed 800, and may even be as high as 20,000. These post-transcriptional regulators of gene expression in higher eukaryotes play an important role in development, tumor suppression, and other cellular processes by hybridizing to complementary target messenger RNA (mRNA) transcripts and ultimately down-regulating or up-regulating gene expression depending on the abundance of that particular miRNA. Because of this unique function, special attention has been given to miRNAs as candidate drug targets for cancer, diabetes, obesity, and viral diseases, wherein miRNAs influence cancer development by serving as either tumor suppressors or oncogenes, but the regulation of miRNA is poorly investigated.

While miRNAs have been shown to affect post-transcriptional regulation, regulation of developmental timing and pattern formation, restriction of differentiation potential, regulation of insulin secretion, resistance to viral infection, genomic rearrangements associated with carcinogenesis and other genetic disorders, the role of miRNAs and other ncRNAs with respect to melanoma development or progression is not understood. In addition, miRNA target genes within melanomas have remained undiscovered. Therefore an important unmet need exists to further identify and characterize biomarkers including miRNAs and other ncRNAs that contribute to the molecular signatures for melanoma, to identify epigenetic changes in miRNA and other ncRNA expression that contribute to melanoma pathogenesis, and for the development of diagnostic and prognostic methods recognizing these signatures for efficient testing, diagnosis, and treatment of melanoma.

### SUMMARY

Described herein are methods for evaluating the risk and/or progression of melanoma in subjects. The methods involve detecting and quantifying one or more biomarkers associated with melanoma in a biological sample from a subject. The biomarkers useful in predicting the risk of melanoma are also described in detail. The advantages of the invention will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing and general description and the following detailed description are exemplary and explanatory only and are not restrictive.

### BRIEF DESCRIPTION OF FIGURES

The accompanying Figures, which are incorporated in and constitute a part of this specification, illustrate several aspects described below.

FIG. 1 shows differentially expressed miRNA in melanoma cell line WM1552C compared to normal melanocyte cell line HEM-1.

FIG. 2 shows various miRNA expression levels in six different melanoma cell lines and melanocytes by qRT-PCR.

FIG. 3 shows Northern blotting analysis of three miRNAs (miR-211, miR-16, and let-7g) in melanoma cell lines and melanocytes.

FIG. 4 shows the quantification of miR-34b expression in different grades of melanoma, normal skin, and melanocytes by qRT-PCR.

FIG. 5 shows miR-211 target gene expression in melanoma compared to melanocytes.

FIG. 6 shows miR-16 target gene expression in melanoma compared to melanocytes.

FIG. 7 shows miR-34b target gene expression in melanoma compared to melanocytes.

FIG. 8 shows quantification of KCNMA1 target gene expression under various conditions.

FIG. 9 shows a Western Blot of KCNMA1 target gene expression under various conditions.

FIG. 10 shows differential expression among melanoma cell lines with a stably integrated miRNA expressing construct versus cell lines without an miRNA expressing construct.

FIG. 11 shows an example construct for β-galactosidase target cleavage assays.

FIG. 12 shows β-galactosidase target cleavage assay for miR-211 and its target TCF-12.

FIG. 13 shows β-galactosidase target cleavage assays for miR-211 and its targets RAB22A and SLC37A3.

FIG. 14 shows a differential expressing of miRNAs in melanoma cell lines treated with 5AzadC, 4PBA, or a combination of 5AzadC and 4PBA.

FIGS. 15 and 16 show miR-375 and miR-34b CpG island methylation respectively in WM1552C.

FIG. 17 shows CpG island methylation in patient samples, normal skin and nevus of the miR-34b putative promoter.

FIG. 18 shows Northern blotting analysis of miR-34b expression in WM1552C when treated with varying doses of 5AzadC.

FIG. 19 shows systems level pathway mapping of both direct and indirect miR-34b putative target genes

FIG. 20 shows systems level pathway mapping of direct miR-34b putative target genes.

FIG. 21 shows qRT-PCR data confirming that NOTCH 1 expression is down regulated in WM1552C/34b cells and melanocytes when compared WM1552C which under express miR-34b.

FIG. 22 shows Western blot analysis confirming that NOTCH1 is up-regulated in WM1552C cells when compared to melanocytes and WM1552C/34b cells.

FIG. 23 shows an ncRNA array for melanoma cell lines, melanocytes, and keratinocytes.

FIG. 24 shows an ncRNA array for patients having melanoma.

### DETAILED DESCRIPTION

Before the present compounds, compositions, and/or methods are disclosed and described, it is to be understood that the aspects described below are not limited to specific compounds, synthetic methods, or uses as such may, of course, vary. It is also to be understood that the terminology used herein is for the purpose of describing particular aspects only and is not intended to be limiting.

In this specification and in the claims that follow, reference will be made to a number of terms that shall be defined to have the following meanings:

It must be noted that, as used in the specification and the appended claims, the singular forms “a,” “an” and “the” include plural referents unless the context clearly dictates otherwise. Thus, for example, reference to “a biomarker” includes mixtures of two or more such biomarkers, and the like.

“Optional” or “optionally” means that the subsequently described event or circumstance can or cannot occur, and that the description includes instances where the event or circumstance occurs and instances where it does not.

“Risk” may be used to refer to a subject that may develop melanoma at a future date. To be at “risk,” the subject's skin may be phenotypically evaluated for abnormalities. Subjects who appear to be phenotypically normal or abnormal may be at “risk” and therefore may be evaluated by the methods described below.

“Progression” refers to a subject suspected to have or diagnosed with melanoma and the various stages of tumor progression associated with melanoma. The methods described herein may be performed over different time intervals ranging from days, weeks, months, and years to evaluate melanoma progression in a subject.

“Biomarker” may be used to refer to a naturally occurring biological molecule present in a subject at varying concentrations useful in predicting the risk or incidence of melanoma. For example, the biomarker can be an miRNA present in higher or lower amounts in a subject at risk for melanoma. The biomarker can include nucleic acids, ribonucleic acids, or a polypeptide used as an indicator or marker for melanoma in a cell, tissue, or subject.

The term “peptide” may be used to refer to a natural or synthetic molecule comprising comprising two or more amino acids linked by the carboxyl group of one amino acid to the alpha amino group of another. The peptide is not limited by length, and thus “peptide” can include polypeptides and proteins.

The term “nucleic acid” may be used to refer to a natural or synthetic molecule comprising a single nucleotide or two or more nucleotides linked by a phosphate group at the 3′ position of one nucleotide to the 5′ end of another nucleotide. The nucleic acid is not limited by length, and thus the nucleic acid can include deoxyribonucleic acid (DNA) or ribonucleic acid (RNA).

“Subject” refers to a mammal, including humans, who are at risk for or have melanoma and benefits from the methods described herein.

“Diagnostic” means identifying the presence or nature of a pathologic condition.

“Detect” refers to the qualitative measurement of undetectable, low, normal, or high concentrations of one or more biomarkers such as, for example, nucleic acids, ribonucleic acids, or polypeptides and other biological molecules.

“Quantify” and “quantification” may be used interchangeably, and refer to a process of determining the quantity or abundance of a substance in a sample (e.g., a biomarker), whether relative or absolute. For example, quantification may be determined by methods including but not limited to, micro-array analysis, qRT-PCR, band intensity on a Northern blot, or by various other methods know in the art.

“About” is used to provide flexibility to a numerical range endpoint by providing that a given value may be “slightly above” or “slightly below” the endpoint without affecting the desired result.

“non-coding RNA or ncRNA” refers to RNA which is not translated into a protein. Examples of ncRNAs include smallRNA (sRNA), non-protein-coding RNA (npcRNA), non-messenger RNA (nmRNA), functional RNA (fRNA), microRNA (miRNA), and small interfering RNA (siRNA).

“miRNA” refers to a single-stranded RNA molecule of about 20-25 nucleotides in length which regulates gene expression. Throughout the text, miRNA, miR, and microRNA may be used interchangeably.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within the ranges as if each numerical value and sub-range is explicitly recited. As an illustration, a numerical range of “about 1 to 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc. as well as 1, 2, 3, 4, and 5, individually. The same principle applies to ranges reciting only one numerical value as a minimum or a maximum. Furthermore, such an interpretation should apply regardless of the breadth of the range or the characteristics being described.

Described herein are methods for evaluating the risk and progression of melanoma in subjects. The methods involve quantifying one or more biomarker(s) associated with melanoma in a biological sample from a subject. Such biomarkers may allow for a prognostic or diagnostic distinction between melanoma and other conditions. Early identification of subjects at risk for melanoma would be of considerable value, as such subjects could be more closely monitored and treated before developing metastatic melanoma.

Testing using the methods described herein may occur at any time when biomarkers indicative of melanoma are quantifiable in the subject. For example, in one aspect a subject that phenotypically appears to have “normal” skin may be tested for melanoma using the methods described herein. In another aspect, a subject that phenotypically appears to have “abnormal” skin may be tested for melanoma. Abnormalities may include skin lesions, discolored moles (nevus) or discolored skin, persistent itching in a skin lesion, change in size, shape, or color of a lesion, ulceration, bleeding, and/or tenderness of the skin, a skin lesion or skin lesions with irregular borders, or any signs or symptoms that a clinician would deem worthy of diagnostic testing.

To quantify whether a biomarker indicative of melanoma is quantifiable in a subject, a biological sample must be acquired. In one aspect, the biological sample includes a biopsy, a skin biopsy, a mole or nevus biopsy, blood, serum, cultured cells including primary and secondary (i.e. immortalized) cultured cells, or any combination thereof.

Biomarkers useful for identifying subjects at risk for or for monitoring the progression of melanoma include non-coding RNAs (ncRNA). Quantification of one or more of these ncRNAs provides some indication of the risk or progression of melanoma for the subject, and thus may provide opportunities for preventative treatments. It should be noted that any biomarker that is predictive of evaluating the risk or progression of melanoma should be considered within the scope of the claims of the present invention. In one aspect, however, nonlimiting examples of biomarkers associated with melanoma may include ncRNAs found to be statistically different (i.e. p<0.01, p<0.02, p<0.05) from control subjects (i.e. biopsies from “normal” subjects that do not have melanoma or primary cell lines derived from “normal” subjects). In another aspect, nonlimiting examples of biomarkers associated with melanoma may include ncRNA found to be qualitatively different from control subjects. In this aspect, qualitative data reflects that a biomarker is often associated with melanoma; however, due to sample size or various other limitations, statistical significance has not been shown.

In one aspect, nonlimiting examples of ncRNAs associated with the risk or progression of melanoma may include miRNAs. In this aspect, these miRNAs may include the nucleotide sequences of UUCCCUUUGUCAUCCUUCGCCU SEQ ID NO 1 (hsa-miR-211) (hereinafter hsa-miR and miR are used interchangeably), CAAUCACUAACUCCACUGCCAU SEQ ID NO 2 (hsa-miR-34b), UUUGUUCGUUCGGCUCGCGUGA SEQ ID NO 3 (hsa-miR-375), UUCCCUUUGUCAUCCUAUGCCU SEQ ID NO 4 (hsa-miR-204), AACCCGUAGAUCCGAUCUUGUG SEQ ID NO 5 (hsa-miR-99a), UAGCAGCACGUAAAUAUUGGCG SEQ ID NO 6 (hsa-miR-16), UGAGGUAGUAGGUUGUAUAGUU SEQ ID NO 7 (hsa-miR-let-7a), UGAGGUAGUAGGUUGUGUGGUU SEQ ID NO 8 (hsa-miR-let-7b), UGAGGUAGUAGGUUGUAUGGUU SEQ ID NO 9 (hsa-miR-let-7c), AGAGGUAGUAGGUUGCAUAGUU SEQ ID NO 10 (hsa-miR-let-7d), UGAGGUAGGAGGUUGUAUAGUU SEQ ID NO 11 (hsa-miR-let-7e), UGAGGUAGUAGAUUGUAUAGUU SEQ ID NO 12 (hsa-miR-let-7f), UGAGGUAGUAGUUUGUACAGUU SEQ ID NO 13 (hsa-miR-let-7g), UGAGGUAGUAGUUUGUGCUGUU SEQ ID NO 14 (hsa-miR-let-7i), UCCCUGAGACCCUUUAACCUGUGA SEQ ID NO 15 (hsa-miR-125a), UCCCUGAGACCCUAACUUGUGA SEQ ID NO 16 (hsa-miR-125b), UAGCAGCACAUCAUGGUUUACA SEQ ID NO 17 (hsa-miR-15b), CCCAGUGUUCAGACUACCUGUUC SEQ ID NO 18 (hsa-miR-199a), UAGCUUAUCAGACUGAUGUUGA SEQ ID NO 19 (hsa-miR-21), ACAGCAGGCACAGACAGGCAGU SEQ ID NO 20 (hsa-miR-214), AGCUACAUUGUCUGCUGGGUUUC SEQ ID NO 21 (hsa-miR-221), AGCUACAUCUGGCUACUGGGU SEQ ID NO 22 (hsa-miR-222), AUCACAUUGCCAGGGAUUUCC SEQ ID NO 23 (hsa-miR-23a), AUCACAUUGCCAGGGAUUACC SEQ ID NO 24 (hsa-miR-23b), CCUAUUCUUGGUUACUUGCACG SEQ ID NO 25 (hsa-miR-26a), UGUAAACAUCCUACACUCUCAGC SEQ ID NO 26 (hsa-miR-30c), AAAAGCUGGGUUGAGAGGGCGA SEQ ID NO 27 (hsa-miR-320), any nucleic acid sequence or ribonucleic acid sequence having between 90 to 100% homology, or any combination thereof.

In yet another aspect, nonlimiting examples of ncRNAs associated with the risk or progression of melanoma may include the following genomic DNA sequences which code for the ncRNA nucleotide sequences: SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, any nucleic acid or ribonucleic acid sequence having between 90 to 100% homology, or any combination thereof.

Various molecular biology and bioinformatics approaches may be used to identify and quantify biomarkers associated with the risk and progression of melanoma. Such molecular biology and bioinformatics approaches include, but are not limited to, ncRNA arrays, miRNA arrays, RT-PCR, qRT-PCR, Northern blotting, Western blotting, DNA sequencing, dideoxy DNA sequencing, bisulfite DNA sequencing, computational mapping for target genes of the above identified ncRNAs and miRNAs, or any combination thereof. For example, without wishing to be bound by theory, standard analysis methods such as delta_delta Ct method, normality of qRT-PCR data and statistical identification of potential outlier values may be accomplished empirically with Box and Whisker plots, and analytically by the Grubbs test when quantifying these biomarkers with qRT-PCR. In this example, a triangulation approach identifies non-coding RNA deemed important for validation by qRT-PCR. This will include statistical significance testing, empirical assessment via fold change, and functional relevancy as measured by enrichment of Gene Ontology terms by Fisher's F-test with Bonferroni correction. These results may be further confirmed by reporter gene assays, stably transfecting ncRNA constructs to restore “normal” ncRNA amounts, or treating cells or tissues with various bioactives and therapeutic agents. The Examples section contains further detail regarding the use of these approaches within the scope of this application.

In one aspect, a method for evaluating the risk or progression of melanoma in a subject may include quantifying the amount of at least one biomarker present in a biological sample derived from the subject. In this aspect, the biomarker may include SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-16), SEQ ID NO 7 (miR-let-7a), SEQ ID NO 8 (miR-let-7b), SEQ ID NO 9 (miR-let-7c), SEQ ID NO 10 (miR-let-7d), SEQ ID NO 11 (miR-let-7e), SEQ ID NO 12 (miR-let-7f), SEQ ID NO 13 (miR-let-7g), SEQ ID NO 14 (miR-let-7i), SEQ ID NO 15 (miR-125a), SEQ ID NO 16 (miR-125b), SEQ ID NO 17 (miR-15b), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof. In this aspect, the amount of the at least one biomarker may be compared to the amount of at least one biomarker in a control biological sample derived from a subject not having melanoma to identify an increased risk or progression of melanoma. To further illustrate, when measuring the amounts of miRNAs in a subject either having melanoma or at risk for melanoma versus a control subject, various miRNAs may be up-regulated or down-regulated. For example, the methods described herein reveal that miR-211 (SEQ ID NO 1) is typically down-regulated (FIG. 1, p<0.02) in melanoma. Therefore, in this aspect, a subject having significantly down-regulated miR-211 when compared to a control subject may be deemed to either be at risk for developing melanoma or already have melanoma. In a further aspect, miR-34b (SEQ ID NO 2) present in a biological sample may be quantified and compared to the amount of at least one biomarker in a control biological sample to further assess the risk or progression of melanoma in a subject. In yet another aspect, miR-375 (SEQ ID NO 3) present in a biological sample may be quantified and compared to the amount of at least one biomarker in a control biological sample to further assess the risk or progression of melanoma in a subject.

In some instances, up-regulation or down-regulation of these ncRNAs, which include miRNAs, may be attributed to a modification in epigenetic regulation of gene expression. Epigenetic regulation may be attributed to, for example, DNA methylation or chromatin remodeling. To determine whether the up-regulation or down-regulation of any ncRNAs was attributed to changes in epigenetic regulation, methylation of CpG islands within the putative promoter regions of the SEQ IDs mentioned above were assayed and samples were further treated with 5AzadC, a methyltransferase inhibitor. Epigenetic regulation is discussed further within the Examples section. Likewise, specific methods and parameters for detecting and quantifying the biomarkers described herein are further provided in the Examples.

As described above, numerous biomarkers have been identified to evaluate the risk or progression of melanoma. Depending on the specific biomarker, a biomarker may either be up-regulated or down-regulated. Biomarkers which include SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 38, SEQ ID NO 39, SEQ ID NO 41, SEQ ID NO 42, SEQ ID NO 43, SEQ ID NO 44, SEQ ID NO 45, SEQ ID NO 47, SEQ ID NO 49, SEQ ID NO 50, SEQ ID NO 53, SEQ ID NO 55, SEQ ID NO 56, SEQ ID NO 58, SEQ ID NO 59, SEQ ID NO 63, SEQ ID NO 65, SEQ ID NO 68, SEQ ID NO 69, SEQ ID NO 70, SEQ ID NO 76, SEQ ID NO 77, SEQ ID NO 78, SEQ ID NO 80, SEQ ID NO 81, SEQ ID NO 82, SEQ ID NO 83, SEQ ID NO 85, SEQ ID NO 87, SEQ ID NO 88, SEQ ID NO 89, or any combination thereof may be down-regulated in a subject at risk for or having melanoma. Specifically, SEQ ID NO 1-5 are miRNAs that target specific mRNAs and subsequently regulate gene expression. Without wishing to be bound by theory, if miRNAs are under expressed or down-regulated, the corresponding target mRNAs will be translated in an increased amount compared to the control subject. In this aspect, either a down-regulation for a particular miRNA can be assayed or the amount of both the target mRNA and the subsequently translated protein can be assayed by northern and western blotting. The results from these assays can be compared with a control subject and the evaluation of risk for or the progression of melanoma can be further assessed.

In another aspect, biomarkers may be up-regulated. For example, these biomarkers include SEQ ID NO 6 (miR-16), SEQ ID NO 7 (miR-let-7a), SEQ ID NO 8 (miR-let-7b), SEQ ID NO 9 (miR-let-7c), SEQ ID NO 10 (miR-let-7d), SEQ ID NO 11 (miR-let-7e), SEQ ID NO 12 (miR-let-7f), SEQ ID NO 13 (miR-let-7g), SEQ ID NO 14 (miR-let-7i), SEQ ID NO 15 (miR-125a), SEQ ID NO 16 (miR-125b), SEQ ID NO 17 (miR-15b), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 40, SEQ ID NO 46, SEQ ID NO 48, SEQ ID NO 51, SEQ ID NO 52, SEQ ID NO 54, SEQ ID NO 57, SEQ ID NO 60, SEQ ID NO 61, SEQ ID NO 62, SEQ ID NO 64, SEQ ID NO 66, SEQ ID NO 67, SEQ ID NO 71, SEQ ID NO 72, SEQ ID NO 73, SEQ ID NO 74, SEQ ID NO 75, SEQ ID NO 79, SEQ ID NO 84, SEQ ID NO 86, or any combination thereof may be up-regulated in a subject at risk for or having melanoma Like SEQ ID NO 1-5, SEQ ID NO 6-27 are miRNAs which target specific mRNAs and subsequently regulate gene expression. Without wishing to be bound by theory, if miRNAs are up-regulated, the corresponding target mRNAs will be translated in a decreased amount compared to a control subject. In this aspect, either an up-regulation for a particular miRNA can be assayed or the amount of both the target mRNA and the subsequently translated protein can be assayed by northern and western blotting. The results from these assays can be compared with a control subjects and a determination of whether a subject is at risk for or has melanoma can be further assessed.

In another aspect, multiple biomarkers may be detected and quantified to evaluate the risk or progression of melanoma. In one aspect, at least two ncRNAs having the sequence SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-let-7a), SEQ ID NO 7 (miR-let-7b), SEQ ID NO 8 (miR-let-7c), SEQ ID NO 9 (miR-let-7d), SEQ ID NO 10 (miR-let-7e), SEQ ID NO 11 (miR-let-7f), SEQ ID NO 12 (miR-let-7g), SEQ ID NO 13 (miR-let-7i), SEQ ID NO 14 (miR-125a), SEQ ID NO 15 (miR-125b), SEQ ID NO 16 (miR-15b), SEQ ID NO 17 (miR-16), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 0 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof are quantified, and a determination of whether a subject is at risk for or has melanoma can be further assessed. In this aspect, SEQ ID NO 1 (miR-211) and SEQ ID NO 2 (miR-34b) present in a biological sample can be quantified and compared to the amount of at least one biomarker in a control biological sample to further assess the risk or progression of melanoma in a subject. Likewise, SEQ ID NO 1 (miR-211) and SEQ ID NO 3 (miR-375) present in a biological sample can be quantified and compared to the amount of at least one or both biomarkers in a control biological sample to further assess the risk or progression of melanoma in a subject. Furthermore, SEQ ID NO 2 (miR-34b) and SEQ ID NO 3 (miR-375) present in a biological sample can be quantified to further assess the risk or progression of melanoma in a subject.

In yet another aspect, at least three ncRNAs having the sequence SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-let-7a), SEQ ID NO 7 (miR-let-7b), SEQ ID NO 8 (miR-let-7c), SEQ ID NO 9 (miR-let-7d), SEQ ID NO 10 (miR-let-7e), SEQ ID NO 11 (miR-let-7f), SEQ ID NO 12 (miR-let-7g), SEQ ID NO 13 (miR-let-7i), SEQ ID NO 14 (miR-125a), SEQ ID NO 15 (miR-125b), SEQ ID NO 16 (miR-15b), SEQ ID NO 17 (miR-16), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof can be quantified, and a determination of whether a subject is at risk of has melanoma can be further assessed. In this aspect, SEQ ID NO 1 (miR-211), SEQ ID NO 2 (miR-34b), and SEQ ID NO 3 (miR-375) present in a biological sample can be quantified to further assess the risk for or the progression of melanoma in a subject.

In a further aspect, at least four ncRNAs having the sequence SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-let-7a), SEQ ID NO 7 (miR-let-7b), SEQ ID NO 8 (miR-let-7c), SEQ ID NO 9 (miR-let-7d), SEQ ID NO 10 (miR-let-7e), SEQ ID NO 11 (miR-let-7f), SEQ ID NO 12 (miR-let-7g), SEQ ID NO 13 (miR-let-7i), SEQ ID NO 14 (miR-125a), SEQ ID NO 15 (miR-125b), SEQ ID NO 16 (miR-15b), SEQ ID NO 17 (miR-16), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof can be quantified to further assess the risk for or the progression of melanoma in a subject.

In another aspect, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, and 89 ncRNAs having the sequence provided by SEQ ID NOs 1-89 may be quantified to further assess the risk for or the progression of melanoma in a subject.

As discussed above and within the Examples section, ncRNAs and particularly miRNAs regulate gene expression of specific target genes. To determine miRNA target genes, computational resources including miRanda, miRbase, miRNAmap, Tarbase, PicTar, Target ScanS, and DIANA MicroTest (http://www.ncrna.org) may be used. First degree interaction maps from each putative target gene are generated to computationally evaluate whether the putative target genes are biologically significant. If deemed to be biologically significant, these targets may be further evaluated.

In one aspect, computational mapping predicted TCF12, RAB22A, SLC37A3 and KCNMA1 as possible targets for miR-211. In addition, computational mapping predicted that FAM38B, COL12A1, VEGFA, IQGAP2, CDK6, DNM1L, ARID4B, THRB, HS3ST3B1, STC1, ARRDC3, DLL1, KCNMA1, NAV3, PCF11, TCF12, TOX, ATXN1, NFAT5, NOTCH1, VEZT, ITSN1, PRR3, TRPS1, PDGFRA, NAV1, C21orf66, RDX, FOXP1, and MYB as possible targets for miR-34b. Computational mapping may be used for any of the SEQ ID NOs listed above to determine miRNA target genes.

In another aspect, these computationally predicted target genes may be confirmed via, microarrays, qRT-PCR, or reporter gene assays. For example, in this aspect, the predicted target of a particular miRNA may be amplified by techniques which include PCR and these amplified predicted targets may be cloned into a plasmid having a reporter gene. In this aspect, the target may be cloned within the 3′ end of the reporter gene to create a fusion gene having a reporter gene-target gene. Furthermore, the reporter gene may include the lacZgene which produces β-galactosidase, a gene encoding luciferase, or a gene encoding green fluorescent protein (GFP). In one aspect, the vector containing the fusion gene may be transfected into cells and fusion gene expression (i.e. β-galactosidase, luciferase, or GFP) may be measured. Without wishing to be bound by theory, endogenous miRNA interacts with the fusion gene's mRNA transcript and initiates gene silencing. If the endogenous miRNA does in fact interact with the predicted target gene, the fusion gene expression levels will vary according to the amount of endogenous miRNA. In another aspect, the vector containing the fusion gene may be contransfected into cells with the miRNA which is being evaluated. This miRNA may comprise RNA or a portion encoding the miRNA may be cloned into a vector and subsequently processed into miRNA upon transfection into the cell. In this aspect, the fusion gene's gene expression may be measured to further confirm whether a predicted target gene is an actual target gene of a particular miRNA. In each of these aspects, one of ordinary skill in the art would readily recognize that these assays may be modified to either measure the increase or decrease of reporter gene expression and could correlate these results to whether a predicted target gene is in fact an actual target of a particular miRNA.

### Examples

The following examples are put forth so as to provide those of ordinary skill in the art with a complete disclosure and description and description of how the compositions, compounds, and methods described and claimed herein are made and evaluated, and are intended to be purely exemplary and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentration, temperatures, pressures, and other reaction ranges and conditions that can be used to optimize the product purity and yield from the described process. Only reasonable and routine experimentation will be required to optimize such process conditions.

### Cell Lines and Clinical Samples

Experimental studies in this manuscript focused upon the use of human epidermal melanocytes (ScienCell, Catalog #2200) (grown in MelM media containing MelGS growth supplements, 0.5% FBS, and penn/strep solution) and the melanoma cell lines A375 (ATCC®Number: CRL-1619), HT-144 (ATCC® Number: HTB-63), RPMI-7951 (ATCC® Number: HTB-66), SK-MEL2 (American Type Culture Collection, Manassas, Va.), WM793B (ATCC® Number: CRL-2806), RPMI-7951 (ATCC® Number: HTB-66), G-361 (ICLC HTL 99001), LOX, and WM1552C (ATCC® Number: CRL-2808) (grown in Complete Tu Media containing a 4:1 mixture of MCDB-153 medium with 1.5 g/L sodium bicarbonate and Leibovitz's L-15 medium with 2 mM L-glutamine, 2% FBS, and 1.68 mM CaCl₂). Cells were cultured at standard conditions.

Information regarding all clinical samples is described in Table 1.

TABLE 1

| Clinical | |
|---|---|
| Sample # | Tumor Type |
| 1 | Nodal |
| | Metastasis |
| 2 | Nodal |
| | Metastasis |
| 3 | Regional |
| | Metastasis |
| 4 | Nodal |
| | Metastasis |
| 5 | Nodal |
| | Metastasis |
| 6 | Regional |
| | Metastasis |
| 7 | Nodal |
| | Metastasis |
| 8 | Nodal |
| | Metastasis |
| 9 | Distant |
| | Metastasis |
| 10 | Primary |
| | Melanoma |
| 11 | Nodal |
| | Metastasis |
| 12 | Nodal |
| | Metastasis |
| 13 | Distant |
| | Metastasis |
| 14 | Primary |
| | Melanoma |
| 15 | Nodal |
| | Metastasis |
| 16 | Nodal |
| | Metastasis |
| 17 | Distant |
| | Metastasis |
| 18 | Distant |
| | Metastasis |
| 19 | Nodal |
| | Metastasis |
| 20 | Nodal |
| | Metastasis |
| 21 | Primary |
| | Melanoma |
| 22 | Primary |
| | Melanoma |
| 23 | Primary |
| | Melanoma |
| 24 | Primary |
| | Melanoma |
| 25 | Distant |
| | Metastasis |
| 26 | Distant |
| | Metastasis |
| 27 | Regional |
| | Metastasis |
| 28 | Regional |
| | Metastasis |
| 29 | Regional |
| | Metastasis |
| 30 | Regional |
| | Metastasis |

miRNA Arrays

The latest miRNA NCode version 2 array (Invitrogen) containing 553 human and 427 mouse miRNAs, as well as the TILDA array (ABI) were used. miRNA was labeled with the AlexaFluor conjugated dendrimers using the direct labeling kit supplied by Genisphere Corp. Hybridization temperatures were routinely evaluated by discriminating between 2 nt variants at internal sites, and most probes can distinguish between 1 nt variants. The arrays were scanned by Axon B-4000. Differentially expressed miRNA genes were identified in the melanoma cell line WM 1552C, a cell line derived from a 72 year old patient with a stage 3 skin melanoma, and compared to a normal melanocyte cell line, HEM-1, by hybridization of total RNA samples isolated from these two cell lines to the NCode and TLDA miRNA arrays. FIG. 1 lists 25 statistically significant, differentially expressed miRNAs. To address whether the differential miRNA expression levels observed within the cell line WM1552C varied among other established melanoma cell lines, quantitative reverse transcriptase mediated polymerase chain reaction (qRT-PCR) analysis was performed on RNA isolated from WM1552C and the seven additional melanoma cell lines mentioned above. Beyond standard analysis methods such as delta_delta Ct method, normality of qRT-PCR data and statistical identification of potential outlier values were accomplished empirically with Box and Whisker plots, and analytically by the Grubbs test. A triangulation approach will identify those non-coding RNA deemed important for validation by qRT-PCR. This will include statistical significance testing, empirical assessment via fold change, and functional relevancy as measured by enrichment of Gene Ontology terms by Fisher's F-test with Bonferroni correction. Specific miRNAs shown in FIG. 1 were selected according to their statistical significance, and three such groups (P values<0.01, 0.02 and 0.05) are illustrated in this figure. In addition, miR-34b and miR-375 were qualitatively shown to be down-regulated within the WM1552C cell line and will be discussed in further detail below (data not shown). Two statistically significant down-regulated miRNAs (miR-211 and miR-204) and three statistically significant up-regulated miRNAs were selected for expression verification.

Validation of miRNA Array Results

Expression levels of all statistically significant and differentially expressed miRNAs (as determined by arrays) were confirmed by miRNA qRT-PCR using Taqman expression kits (Applied Biosystems) using multiple technical and biological replicates. Standard procedures were followed according to the manufacturer's guidelines. RNU48 was the internal reference probe for normalization of expression values. RNA analysis was performed by northern blot using 20 μg of total RNA concentrated from each sample (melanoma cell lines and melanocytes), separated on 15% TBE-Urea-polyacrylamide gels by electrophoresis. Gels were electroblotted to nylon membranes, cross-linked by UV, prehybridized in Ultrahyb-Oligo (Ambion) for 30′ at 42° C. and hybridized with 5′-biotinylated anti-miRNA DNA oligonucleotides (using anti-U6 reference probes) (10 pM each) at 42° C. overnight, washed and detected by chemiluminiscence (Brightstar Detection kit, Ambion).

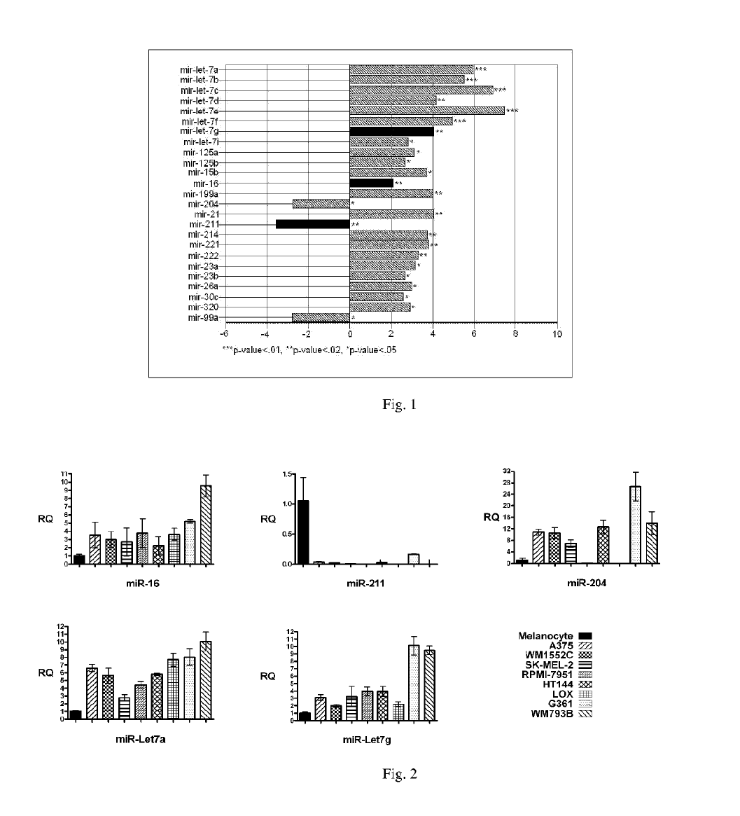

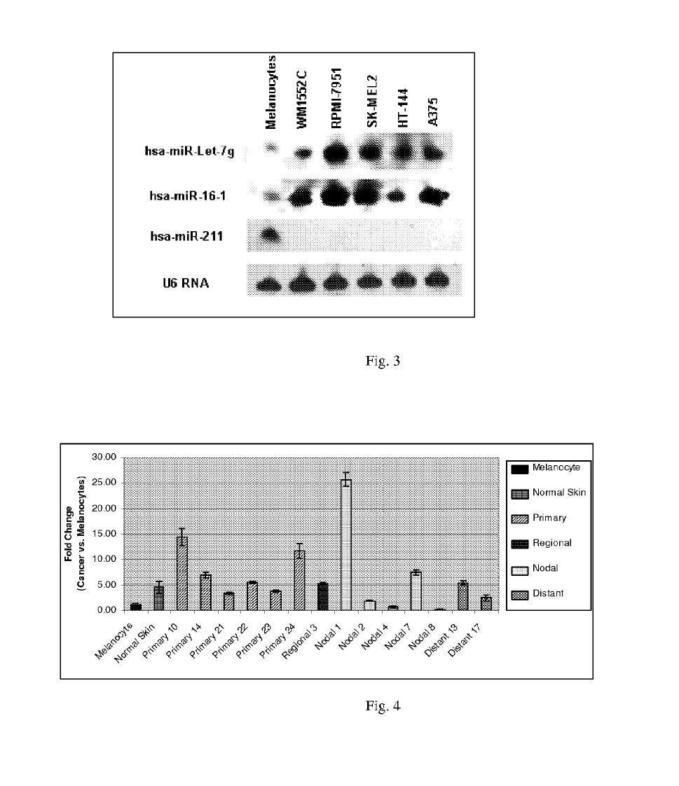

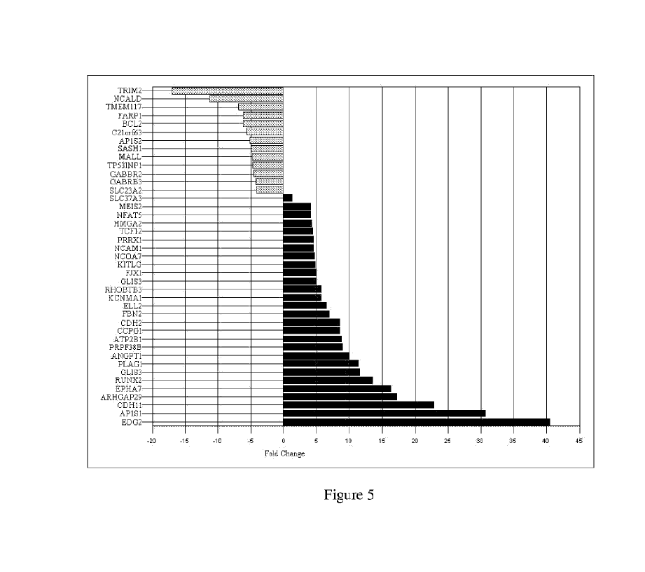

Two down-regulated miRNAs (miR-211 and miR-204) and three up-regulated miRNAs (miR-16, miR-Let7a, and miR-Let7g) were selected for expression verification using qRT-PCR using Taqman expression kits. Among the tested miRNAs, three miRNAs (miR-16, miR-Let7a, miR-Let7g) were expressed at increased levels and one (miR-211) was decreased in all melanoma cell lines compared to those in melanocytes (FIG. 2). While miR-211 was down-regulated in all 7 tested melanoma cell lines, miR-204 was down-regulated only in LOX, RPM1, WM1552C (data not shown). Northern blotting analysis further confirmed the expression levels of miR-16, miR-Let7g, and miR-211 within melanocytes, WM1552C, RPMI-7951, SK-MEL2, HT-144, and A375 (FIG. 3). miR-211 was expressed in melanocytes but not in any of the tested melanoma cell lines.

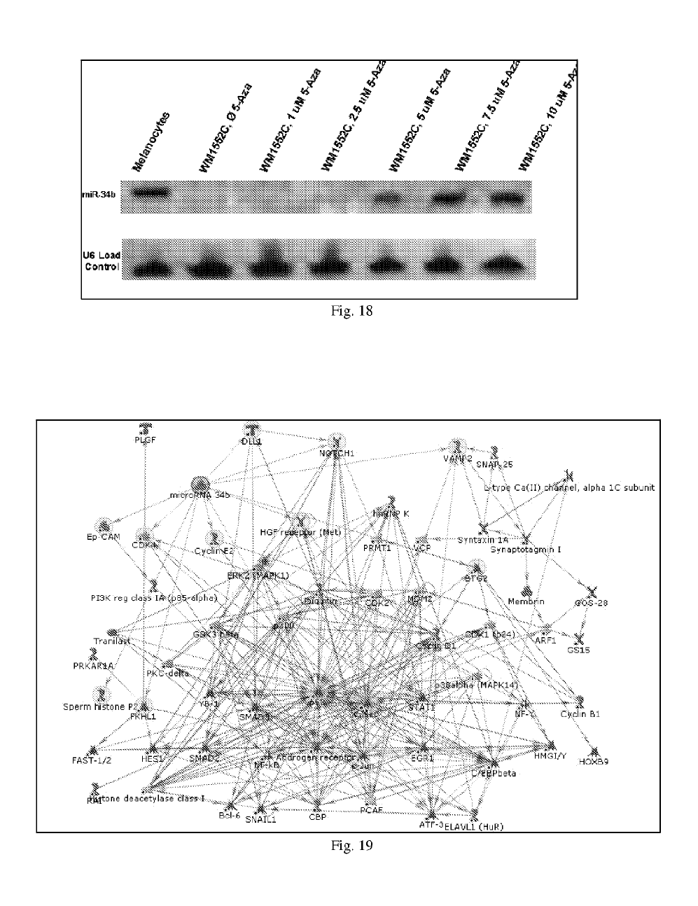

Likewise, miR-34b and miR-375 were qualitatively shown to be down-regulated in WM1552C when compared to melanocytes (see FIG. 18, lanes 2-4 for miR-34b data, data not shown for miR-375). These results were verified by qRT-PCR.

Next, clinical melanoma samples were differentiated from melanocytes according to their levels of endogenous miRNAs. The two miRNAs that showed the most consistent expression level changes in opposite directions were selected; miR-16 was selected for over-expression and miR-211 for under-expression in clinical melanoma samples. miR-211 and miR-16 transcript levels were assayed by qRT-PCR in 30 clinical melanoma samples isolated from skin biopsies, as well as in pre-established cell lines of melanocytes, keratinocytes and normal human skin samples. The level of miR-211 was statistically significantly depleted in 23 of these clinical samples compared to its levels observed in melanocytes (Table 2). In the remaining seven melanomas, five showed statistically significant increases in miR-211 expression, whereas its expression remained unchanged in two melanoma samples. The level of miR-16 was increased in 10 clinical samples but was significantly decreased in 9 samples. In addition, miR-34b expression in different grades of melanoma, normal skin, and melanocytes was quantified by qRT-PCR (FIG. 4). FIG. 4 illustrates the miRN-34 expression in melanocytes, normal skin and various melanoma samples (primary melanoma, regional metastasis melanoma, distant metastasis melanoma, and nodular metastasis melanoma). Unlike cultured melanoma cell lines, patient samples are made out of many different cells. Therefore, expression of a given miRNA varies depending on the site of the origin of melanoma, disease stage, patient's age, gender, different processes in melanoma development and progression, or individual genetic differences.

miR-211 was down-regulated in the majority of melanoma samples. However, there were several exceptions such as the miR-211 up-regulation in one primary, one regional, two distant, and two nodal metastasis clinical melanoma samples. miR-211 is also reduced in normal skin samples, which is expected because melanocytes constitute a minor fraction of cells. These results suggest that development of most melanoma, which is thought to originate in melanocytes, may specifically involve depletion of miR-211 transcript levels.

TABLE 2

| miR-211 | | | miR-16 | |
|---|---|---|---|---|
| Sample Name | Avg RQ | RQ St Dev | Avg RQ | RQ St Dev |
| Melanocyte^{‡} | 1.00063 | 0.04371 | 1.00026 | 0.02824 |
| Normal Skin 1 | 0.01538 | 0.00330 | 1.29108 | 0.08171 |
| Normal Skin 2 | 0.00272 | 0.00067 | 21.37429 | 2.03094 |
| Normal Skin 3 | 0.07133 | 0.01717 | 1.06501 | 0.04539 |
| Normal Skin 4 | 0.02671 | 0.00515 | 4.41538 | 0.90987 |
| Normal Skin 5 | 0.03778 | 0.00469 | 2.63991 | 0.31561 |
| Mean Normal | 0.01976 | 0.00391 | 3.21330 | 0.29310 |
| Primary MM 10 | 2.20605 | 0.12293 | 2.39985 | 0.27298 |
| Primary MM 14 | 0.00093 | 0.00041 | 1.06966 | 0.13106 |
| Primary MM 21 | 0.03565 | 0.00577 | 1.61324 | 0.13542 |
| Primary MM 22 | 0.00419 | 0.00086 | 0.92716 | 0.03975 |
| Primary MM 23 | 0.00226 | 0.00189 | 1.15155 | 0.02580 |
| Primary MM 24 | 0.52232 | 0.15612 | 1.06756 | 0.04822 |
| Mean Primary | 0.02669 | 0.00647 | 1.29517 | 0.07881 |
| MM | | | | |
| Regional MM 3 | 0.01264 | 0.00628 | 0.89322 | 0.07994 |
| Regional MM 6 | 0.01640 | 0.00033 | 0.52149 | 0.02002 |
| Regional MM 27 | 1.44156 | 0.06496 | 0.50261 | 0.02094 |
| Regional MM 28 | 0.00155 | 0.00016 | 1.59185 | 0.06065 |
| Regional MM 29 | 0.00021 | 0.00006 | 1.25152 | 0.10997 |
| Regional MM 30 | 0.05138 | 0.00201 | 0.14129 | 0.00150 |
| Mean Regional | 0.01309 | 0.00116 | 0.63555 | 0.02635 |
| MM | | | | |
| Distant MM 9 | 0.00095 | 0.00011 | 0.89955 | 0.03178 |
| Distant MM 13 | 3.93166 | 0.07708 | 2.59978 | 0.02748 |
| Distant MM 17 | 0.00774 | 0.00049 | 0.84285 | 0.00337 |
| Distant MM 18 | 0.01958 | 0.00208 | 0.57200 | 0.02262 |
| Distant MM 25 | 1.00047 | 0.09279 | 0.41892 | 0.04076 |
| Distant MM 26 | 2.02149 | 0.13435 | 2.62907 | 0.13364 |
| Mean Distant MM | 0.10219 | 0.00692 | 1.03675 | 0.02670 |
| Nodal MM 1 | 0.04167 | 0.02092 | 0.31073 | 0.02781 |
| Nodal MM 2 | 0.00420 | 0.00047 | 0.82755 | 0.13423 |
| Nodal MM 4 | 1.86626 | 0.03250 | 0.61582 | 0.02134 |
| Nodal MM 5 | 0.00037 | 0.00008 | 1.81097 | 0.02605 |
| Nodal MM 7 | 0.01243 | 0.00095 | 0.19970 | 0.01478 |
| Nodal MM 8 | 0.02438 | 0.00144 | 0.35540 | 0.01480 |
| Nodal MM 11 | 0.00318 | 0.00034 | 1.66730 | 0.06945 |
| Nodal MM 12 | 0.00061 | 0.00012 | 1.19227 | 0.03328 |
| Nodal MM 15 | 1.15988 | 0.04215 | 1.62191 | 0.07544 |
| Nodal MM 16 | 0.55816 | 0.03670 | 0.20353 | 0.01710 |
| Nodal MM 19 | 0.08403 | 0.00731 | 2.25307 | 0.29363 |
| Nodal MM 20 | 0.04442 | 0.00172 | 0.84962 | 0.04865 |
| Mean Nodal MM | 0.02729 | 0.00233 | 0.73673 | 0.04048 |

^{‡}Note: Two-trailed T-test comparisons for mir-211 and mir-16 by mean relative quantification levels of melanocyte and primary in situ, regional, distant, and nodal malignant melanoma were all statistically significant at p-value <.000001, except for mir-16 and distant malignant melanoma with (t-test 3.04, df 10) p-value = .0012).
MM—Metastasis melanoma.

Transfection with miRNA Mimic and RNA Expression Analysis

WM1552C cells were washed with PBS, trypsinized, and resuspended in complete TU Media at 10⁵ cells/mL. Cells (2×10⁵) were transfected using siPORT NeoFX transfection reagent (Ambion) overnight with 10 nM mimic microRNA miR-211 (optimized for viability and expression in the range 0-250 nM) (Dharmacon). Cells were subsequently washed with PBS and incubated in fresh growth media. After 48 h (optimized for viability and expression levels in the range of 0-72 h) cells were recovered by trypsinization. Total RNA was isolated using the PureLink kit (Invitrogen), and qRT-PCR was performed (as above).

To address whether there is a causal relationship of miRNA differential expression in melanoma, predicted target genes of miR-211, miR-16, and miR-34b were determined (discussed below).

Microarray Data Analyses and miRNA Target Prediction

For the initial transformation of miRNA array data, GenePixPro 6.0 global normalization method was employed in which images and results are normalized together. Statistical significance tests were Welsh t-test, nonparametric ANOVA, (e.g., to select genes that have significantly less within sample variance compared to between sample variance), and correlation analysis with Pearson's product moment r and Spearman's r. Analysis was controlled for false discovery rate using q-values, with a priori cut point of 10 percent. For mRNA expression array data, commencing with GeneChip® Human Exon 1.0 ST Array (Affymetrix, Inc.) four probes per exon and roughly 40 probes per gene, 7 total arrays were constructed; three melanocyte, and four melanoma. Cell files were loaded into Partek® Genomics Suite™ (Partek, Inc. St. Louis, Mo., USA) under the following algorithm constraints: interrogating probes selection, RMA background correction, adjusted for GC content, quintile normalization, log probes using base 2, with probe set summarization of median polish. Quality control assessment indicated clear separation based on tissue type. Gene level analysis use an ANOVA model; yⱼ=μ+Tⱼ+ε, where μ is the mean expression of the gene, Tⱼ is the tissue type, and ε is the error term. The ANOVA model generated a significance level for each probe set, along with the fold change, and imputed gene annotations. miR-16 target set of genes were obtained from public databases [miRanda, miRbase, miRNAmap, Tarbase, PicTar, Target ScanS, and DIANA MicroTest (http://www.ncma.org)] and the results from ANOVA were matched to obtain the final target gene list of 54 genes. This target list was imported into Ingenuity Pathway Analysis Version 6.0-1202 (Ingenuity Systems®, Redwood City, Calif., USA). A core analysis was run employing direct relationships only, the Ingenuity knowledge base genes as the reference set, and with down-regulators as the defined expression value parameter. Results showed correlated expression in melanoma cells compared to those in melanocytes. Results for miR-211 target binding yielded TCF12, RAB22A, SLC37A3 and KCNMA1 as possible targets for miR-211. Results for miR-204 target binding were substantially similar and yielded TCF12, RAB22A, SLC37A3, and KCNMA1 as possible targets for miR-204. In addition, results for miR-34b target binding yielded numerous possibilities including FAM38B, COL12A1, VEGFA, IQGAP2, CDK6, DNM1L, ARID4B, THRB, HS3ST3B1, STC1, ARRDC3, DLL1, KCNMA1, NAV3, PCF11, TCF12, TOX, ATXN1, NFAT5, NOTCH1, VEZT, ITSN1, PRR3, TRPS1, PDGFRA, NAV1, C21orf66, RDX, FOXP1, and MYB.

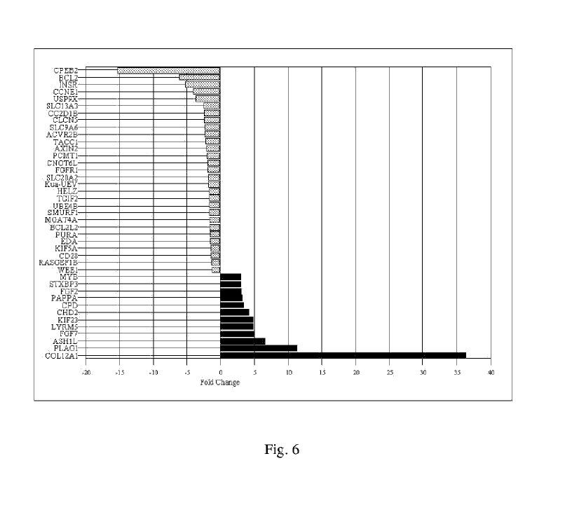

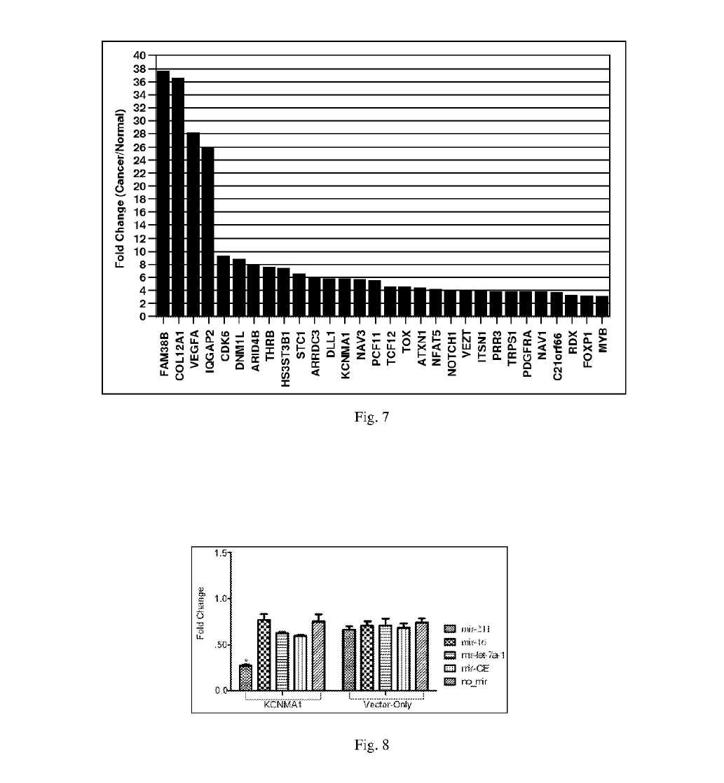

Direct targets of miR-211 were expected to be up-regulated, and those of miR-16 down-regulated, in melanoma cells. First, the top-scoring target genes of miR-211 and miR-16 were selected according to the highest total context score from the target scan database (www.targetscan.org). Second, whole-genome levels of total RNA isolated from melanoma cell line WM1552C and the melanocyte line HEM-1 by hybridizing to Affymetric exon arrays, and filtered the data for differential expression of the top-scoring target genes of miR-211 and miR-16, respectively (FIG. 5 and FIG. 6). Likewise, in melanoma, miR-34b target genes were expected to be up-regulated in comparison to melanocytes. Affymetrix exon array experiments confirmed the up-regulation of these miR-34b target genes (FIG. 7). miR-34b will be discussed in further detail below. Third, to investigate the possible relevance of miR-211 in melanoma, a miR-211 expressing stable WM1552C cell line was engineered. Stable cell lines selected for Zeocin were assayed for the expression of miR-211 by qRT-PCR. After selecting stable cell lines, miR-211 target binding for TCF12, RAB22A, SLC37A3 and KCNMA1 was confirmed by a beta gal target cleavage assay.

Construction of miR-211 Expressing Stable WM1552C Cell Line:

To confirm the miRNA target prediction analyses, a stably expressing miR-211 WM1552 cell line was constructed by random integration of the construct described below. Oligonucleotides complimentary to the hsa-miR-211 genomic sequences (miR-211 pre For-TTCCCTTTGTCATCCTTCGCCT (SEQ ID NO 90) and miR-211 pre Rev-AGGCGAAGGATGACAAAGGGAA (SEQ ID NO 91), containing HindIII and BamHI sites on their respective 5′ and 3′ ends), were used to amplify the 110 bp pre-miR-211 sequence from human melanocyte genomic DNA (Amplitaq Gold, Applied Biosystems) and TOPO-cloned into the pCR4-TOPO vector (Invitrogen). The construct was sequenced and the pre-hsa-miR-211 fragment was sub-cloned into pcDNA4/myc-HisA (Invitrogen) to create pcDNA4/miR-211. 2.5×10⁵ WM1552C Melanoma cells were seeded into a single well of a 6-well plate and transfected overnight with 5 μg pcDNA4/miR-211 using Fugene 6 (Roche). The transfected cells were selected on 800 μg/mL Zeocin for 15 days and the presence of the transgenic copy in stably Zeocin resistant foci was confirmed by PCR (Amplitaq Gold, Applied Biosystems).

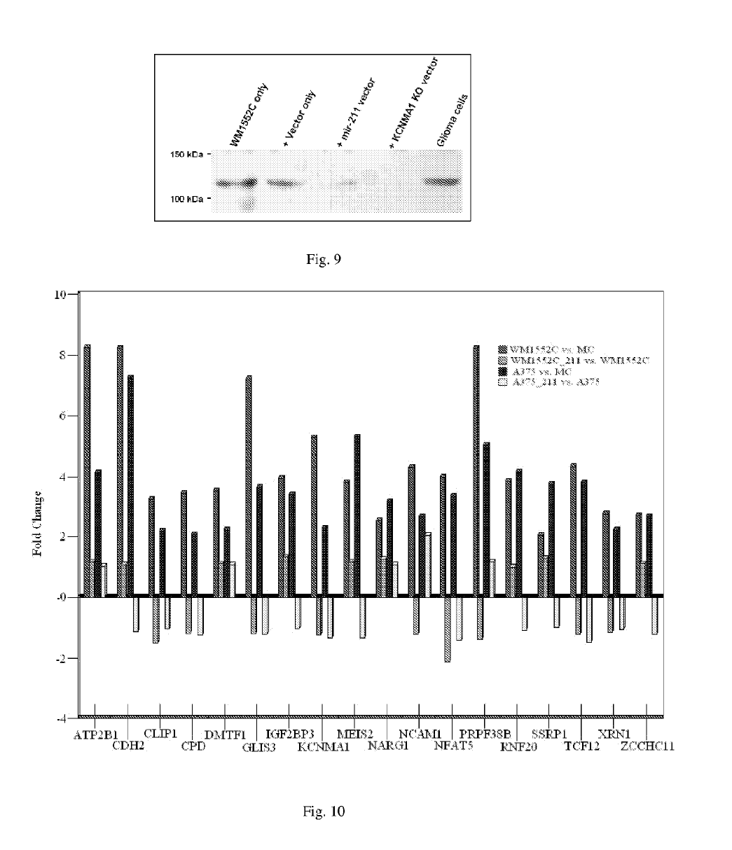

Next, KCNMA1 protein levels were measured in WM1552C only, WM1552C plus the vector only (a negative control), WM1552C plus the stably integrated pcDNA4/miR-211 construct, WM1552C plus KCNMA1 KO vector (a positive control siRNA construct), and glioma cells by western blotting analysis. (FIG. 8 and FIG. 9) FIG. 8 and FIG. 9 confirm that KCNMA1 protein expression is completely abolished by the positive control, WM1552C plus KCNMA1 KO vector, and is partially abolished by WM1552C plus the stably integrated pcDNA4/miR-211 construct. Protein expression of KCNMA1 is highly visible in WM1552C, WM1552C plus the vector only, and in the glioma cells. In addition, FIG. 10 shows ectopic expression in WM1552C and A375 which have stably integrated the pcDNA4/miR-211 construct. Expression levels of various predicted targets were then quantified in comparison to melanocytes, WM1552C cells not having the pcDNA4/miR-211 construct, and A375 cells not having the pcDNA4/miR-211 construct.

### Target Cleavage Assay

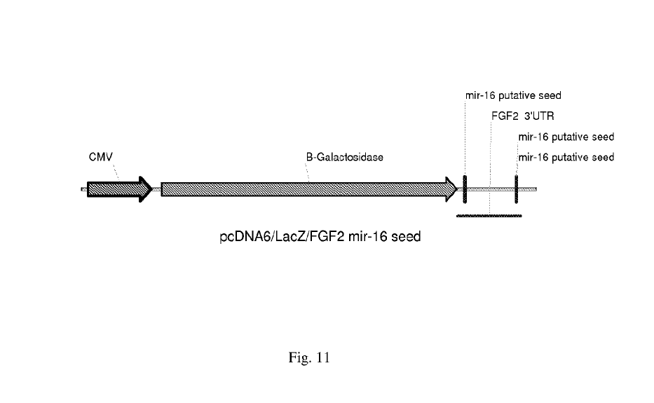

Target cleavage assays using the miRNA targets from the bioinformatics analyses above were conducted. The 3′ UTR seed sequences of putative target genes were amplified by PCR (Phusion PCR kit, Finnzymes) from human Melanocyte genomic DNA (Primers: ARL2 For-ctcctccaccccagcctgc (SEQ ID NO 92), ARL2 Rev-tgagtgaaggatgaggcccacag (SEQ ID NO 93); FGF2 For-cagaagaataggtggtatgttcctaatg (SEQ ID NO 94), FGF2 Rev-gcagcatctgtaagattcttctatctg (SEQ ID NO 95); RAB22A For-taacatttgtaaagggaaaattagcact (SEQ ID NO 96), RAB22A Rev-agtgctaattttccctttacaaatgtta (SEQ ID NO 97); SLC37A3 For-ttactgacaaaaagggaaaatacgaaac (SEQ ID NO 98), SLC37A3 Rev-gtttcgtattttccctttttgtcagtaa (SEQ ID NO 99); TCF12 For-gcaagcagtgtgtcgcttctgcac (SEQ ID NO 100), TCF12 Rev-gcaagaggataggagagggcaac ((SEQ ID NO 101); all primer sets containing 5′ NotI and 3′ AgeI sites). PCR products were cloned into pCR4-TOPO (Invitrogen), confirmed by sequencing, then subcloned into the 3′ UTR of the LacZ gene in pcDNA6/V5-His/LacZ (Invitrogen) using the 5′ NotI and 3′ AgeI restriction sites, and reconfirmed by sequencing (pcDNA6/LacZ/ARL2, pcDNA6/LacZ/FGF2, pcDNA6/LacZ/RAB22A, pcDNA6/LacZ/SLC37A3, and pcDNA6/LacZ/TCF12, respectively). FIG. 11 is an example of the pcDNA6/LacZ construct used within these experiments. As shown, the FGF2 target sequence may be cloned into the 3′ UTR of the LacZ gene. A375 Melanoma cell lines were transfected in triplicate (Fugene 6, Roche) with 5 μg plasmid DNA of: A) one of the five pcDNA6/LacZ/3′ target UTR clones (above), B) pcDNA6/V5-His/LacZ (positive control) or C) no vector (Negative control), and co-transfected (siPORT, Ambion) at 100 nM with miRIDIAN microRNA Mimics (Dharmacon) for A) miR-16, B) miR-211, C) miR-let-7a-1, D) miRIDIAN Negative Control #1. Negative control sequence is based on cel-miR-67, mature sequence: UCACAACCUCCUAGAAAGAGUAGA (SEQ ID NO 102) Cel-miR-67 has been confirmed to have minimal sequence identity with miRNAs in human, mouse and rat or E) no mimic. After overnight incubation, cells were washed in PBS and reincubated in fresh media. After 48 h, cells were harvested by trypsinization, examined for viability, and samples were prepared for beta-galactosidase assay using the β-Gal Assay kit (Invitrogen). Samples were incubated overnight at 37° C., then assayed for beta-galactosidase activity in a 96-well plate format using a FlexStation3 (Molecular Devices).

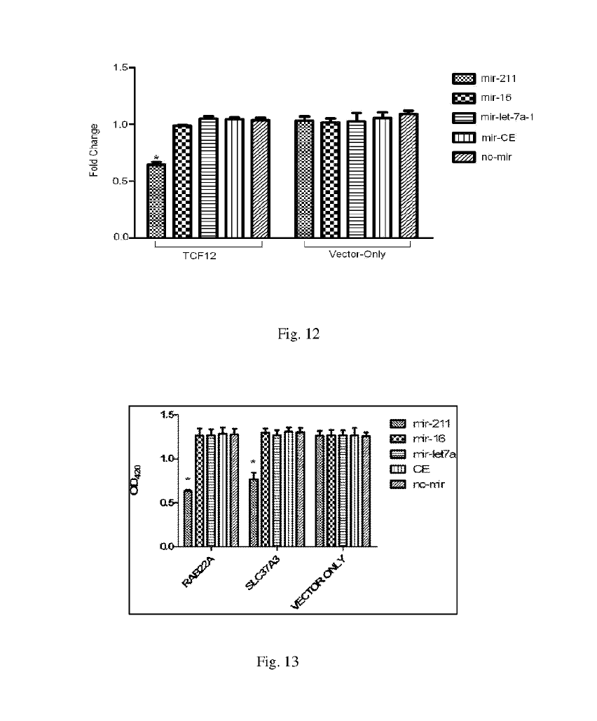

miR-211 target binding in TCF12, RAB22A, SLC37A3 and KCNMA1 was confirmed by a beta gal target cleavage assay. FIG. 12 depicts the β-gal target cleavage assay for miR-211 and its target TCF-12. In this experiment, the miRNA target is cloned into the 3′ end of beta-galactosidase. If the cloned fragment is a target of the co-transfected miRNA, beta-galactosidase expression is expected to be suppressed. However, if the cloned fragment is not a target of the co-transfected miRNA, beta-galactosidase expression will not be suppressed. In FIG. 12, the vector-only, without the TCF-12 fragment, was used as a control for beta-galactosidase activity. In addition, miR-16, Let-7a, and scramble oligo (miR-CE) were used as negative controls. For miR-211, there was a statistically significant reduction in fold change (Kruskal Wallis test, Chi-square=24.142, p-value<0.001).

FIG. 13 depicts miR-211 target cleavage assays with RAB22A and SLC37A3. Each construct was made according to the procedure described above. As with the TCF12 target cleavage assay, beta-galactosidase activity decreased when RAB22A and SLC37A3 were cloned into the 3′ end of the beta-galactosidase gene. These results further confirmed that RAB22A and SLC37A3 mRNA are targets of miR-211.

Likewise, using the construct shown in FIG. 11, target cleavage assays confirmed that FGF2 was in fact a target of miR-16 (data not shown). In this assay, miR-211, miR-let-7a-1, miR-scramble, and no miR were used as negative controls. In addition, the pcDNA6/LacZ vector only and pcDNA6/LacZ/ARL2 were used as controls to confirm that miR-16 specifically targets FGF2, and that miR-16 targeting of FGF2 was not related to an aberrant or random event.

5AzadC Treated Melanoma Cells Reactivate Expression of miR-34b

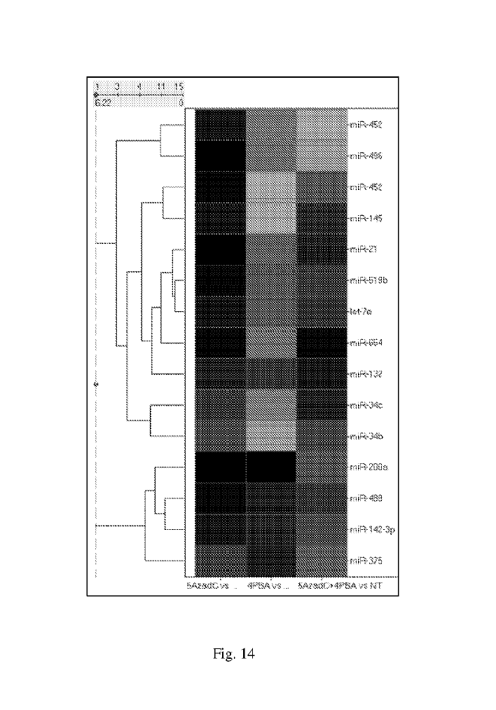

Epigenetic regulation was theorized to be involved in several of the down-regulated miRNAs linked to melanoma. To understand the epigenetic gene regulation of miRNA in melanoma, the melanoma cell line WM1552C (WM1552C) was treated for 24 hrs with 5AzadC, 4-PBA and 5AzadC+4-PBA to identify DNA methylation and histone modification related transcriptional changes. Optimal concentration of 5AzadC and PBA for the treatment was identified by measuring the cell viability and cell survival assays. Three independent biological experiments were conducted with different doses of either 5AzadC or PBA. The higher doses of PBA treatment (5 mM and above) shown to be toxic and demonstrated the cell death. 3 μM-5AzadC and 1 mM-PBA were chosen as optimal concentrations. miRNA expression was measured in treated cells with miRNA commercial arrays (TILDA_ABI and NCode_Invitrogen) as described above. Both arrays confirmed that several miRNAs were up-regulated in WM1552C after the treatment (FIG. 14). FIG. 14 displays hierarchical clustering of differentially expressed miRNAs in melanoma cell line WM1552C treated with 5AzadC, PBA, and 5AzadC+PBA. Up-regulation (red) and down-regulation (green) of miRNAs after treatment compared to untreated cells are shown in red and green respectively. Fold change values were compared to non-treated samples. Table 3 displays corresponding data derived from the arrays described above and shown within FIG. 14.

TABLE 3

| miRNA_ID | 5AzadC_NT | 4PBA_NT | 5AzadC + 4PBA_NT |
|---|---|---|---|
| miR-34b | 5.158631 | −1.872401333 | 5.237561833 |
| miR-489 | 2.064491667 | 3.527152333 | 4.4565325 |
| miR-375 | 4.701135667 | 3.396965333 | 6.4151335 |
| miR-132 | 3.239277333 | 1.033449333 | 3.177381833 |
| miR-142-3p | 2.927757333 | 3.620565667 | 5.399643833 |
| miR-200a | 2.293775 | 2.088957 | 6.089661833 |
| miR-145 | 2.948197333 | −2.280436333 | 1.2459855 |
| miR-452 | 2.043972 | −2.272345 | 0.170035833 |
| miR-21 | 2.124029667 | 0.090113 | 1.3438535 |
| miR-34c | 4.525376333 | −0.795777333 | 3.258946167 |
| miR-452 | 3.18236 | −0.533972 | −3.034523167 |
| miR-496 | 2.291125 | −0.471388 | −1.682155833 |
| let-7e | 2.960471 | 0.157148667 | 1.095689167 |
| miR-654 | 2.422392 | −0.322611333 | 2.5518105 |
| miR-519b | 2.459962333 | 0.208268 | 0.808306833 |

Several of these up-regulated miRNAs such as miR-34b and miR-375 contain CpG islands in their upstream putative regulatory regions. Some of them were previously shown to contain CpG islands in the upstream regions by bioinformatics analysis suggesting that the reactivation is epigenetic.

### CpG Island Methylation Detection

miRNAs that are differentially regulated are likely to carry epigenetic modifications in the CpG islands located in their regulatory elements. These modifications include DNA methylation or histone modification or both. 2 Kb upstream sequences of these potentially epigenetically modulated miRNAs were extracted and their sequences were scanned for the presence of CpG islands using the Methyl Primer express software from the Applied Bio-system. CpG island methylation of two miRNAs (miR-34b and miR-375) were investigated.

To investigate epigenetic miRNA regulation within melanoma, one μg of genomic DNA was treated with sodium bisulfite according to the manufacturers protocol (Zymo). 2 μL of bisulfite-treated genomic DNA was used for bisulfite PCR. PCR was performed using AmpliTaq Gold thermostable polymerase (Applied Biosystems) and the following thermocycling profile: 6 minute hot-start at 95° C.; 35 cycles of 94° C. for 20 seconds, 54° C. for 25 seconds, and 72° C. for 30 seconds; 10 minute extension at 72° C. PCR products were gel purified using the QiaQuick gel extraction kit (Qiagen) and cloned into the pCR4-TOPO vector (Invitrogen). 9 clones for each miRNA candidate were sequenced using M13 primers and the BigDye Terminator kit v1.1 (Applied Biosystems), analyzed on a 3130×1 Genetic Analyzer (Applied Biosystems), and aligned using VectorNTI AlignX (Invitrogen).

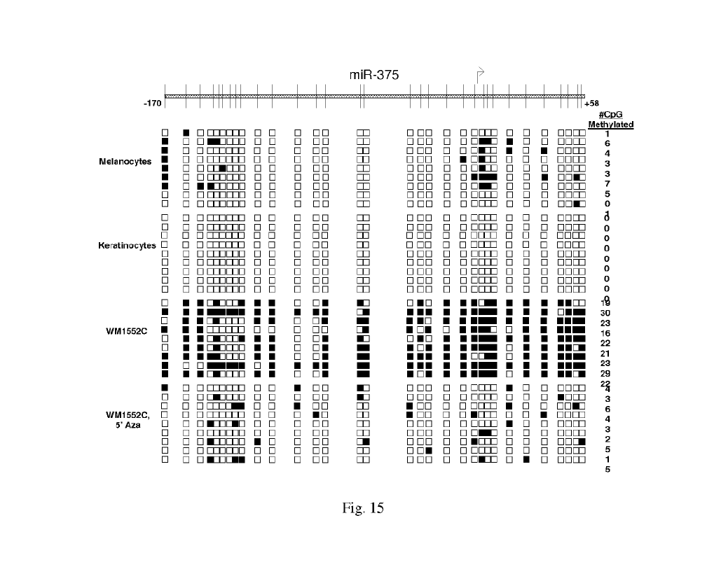

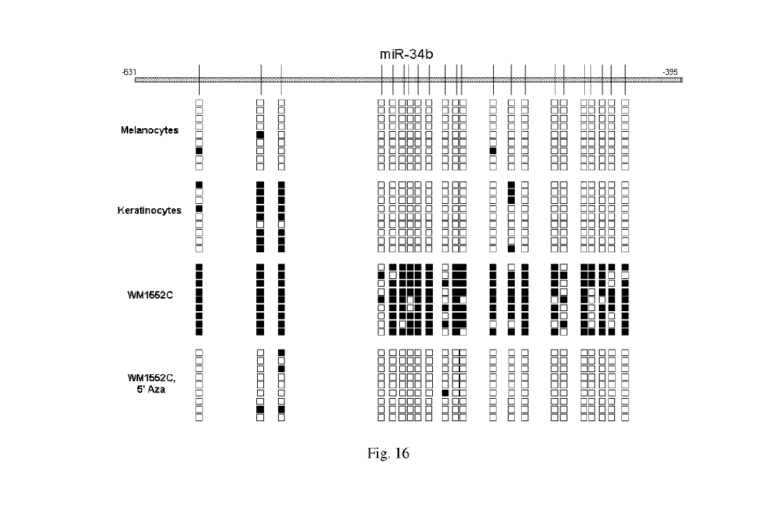

As shown in FIG. 15 and FIG. 16, miR-375 and miR-34b CpG islands are highly methylated in WM1552C, but not in melanocytes or keratinocytes. Specifically, this study focused on the putative promoter region of −631 bps to −396 bps of the CpG islands upstream of miR-34b and focused on the putative promoter region of −170 bps to +58 bps of the CpG islands of miR-375. In addition, miR-124-3a and let-7i were used as negative controls because of the lack of CpG islands (data not shown). No CpG island methylation was detected in either miR-124-3a or let-7i.

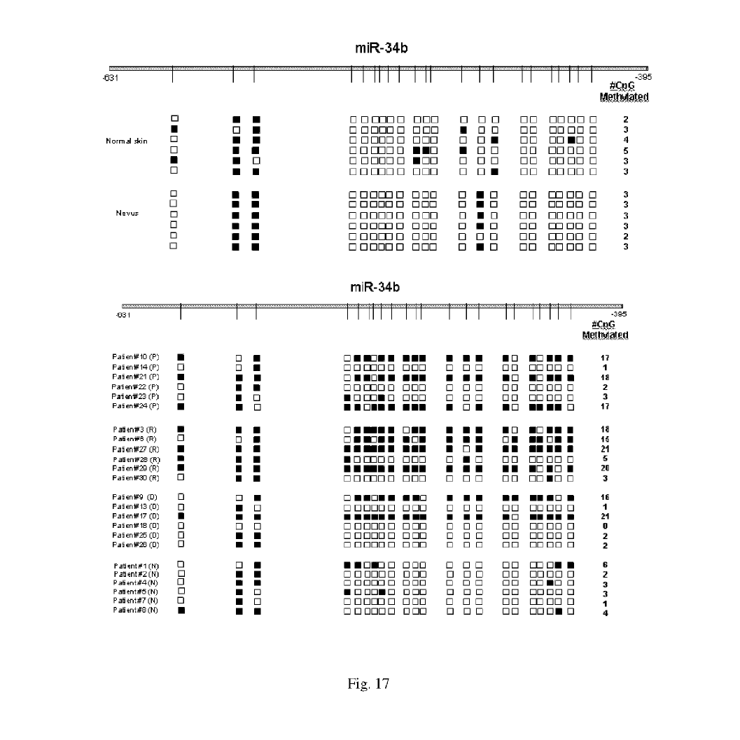

Next, methylation and chromatin modification status of miR-34b was evaluated in a chosen series of melanoma patients. To understand miR-34b expression in melanoma samples, this experiment began with a small group of patients and the qRT-PCR expression was normalized to melanocytes. CpG island methylation in patient samples, normal skin and nevus (i.e. a chronic lesion, a birthmark, or a mole on the skin) of the miR-34b putative promoter was tested and the results are illustrated in FIG. 17. Though the limited number of samples are not conclusive for disease staging or grouping at this time, the presence of CpG island methylation in different stages of patients samples clearly indicate the usefulness of this marker in melanomas. To use miR-34b methylation as an assay for clinical diagnostic testing, it is important to understand the percentage of the CpG island methylation of miR-34b DNA in a given sample. Usually, patient samples are contaminated with melanocytes, keratinocytes, merkel and langrahan unless isolated through laser capture microscopy. Often, these samples contain adipose, fibroblast and other skin cells. Therefore, the best test for DNA methylation studies is the use of DNA purosequencer.

5AzadC Treated Melanoma Cells Reactivate the Expression of miR-34b.

After treating the WM1552C melanoma cells with 5AzadC, a marked increase in the expression levels of several miRNAs was seen in TLDA arrays. These results indicate that differential regulation is epigenetic and that the underlying epigenetic mechanism is CpG island methylation. To support this hypothesis, Northern blots with total RNA obtained from melanoma cell lines which were previously exposed to different concentrations (0.5 μM, 1 μM, 2.5 μM, 5 μM, 7.5 μM and 10 μM) of 5AzadC were run. Subsequent to 5AzadC treatment, miR-34b expression was restored to the level of that in melanocytes. See FIG. 15, FIG. 16, and FIG. 18. No visible expression changes of miR-34b were seen in the low concentrations (1-2.5 μM) of 5AzadC treatment.

miR-34b demethylation results were further confirmed by the CpG island bisulfite studies. See FIG. 15 and FIG. 16. WM1552C treated with 5AzadC completely removed previously methylated sites and this confirms the regulation is epigenetic. See FIG. 15, FIG. 16, and FIG. 18. However, at this point, the origin of transcription start site and any other methylation sites located in the upstream of the tested CpG island miRNA are unknown.

Systems Level Pathway Mapping of miR-34b Putative Target Genes

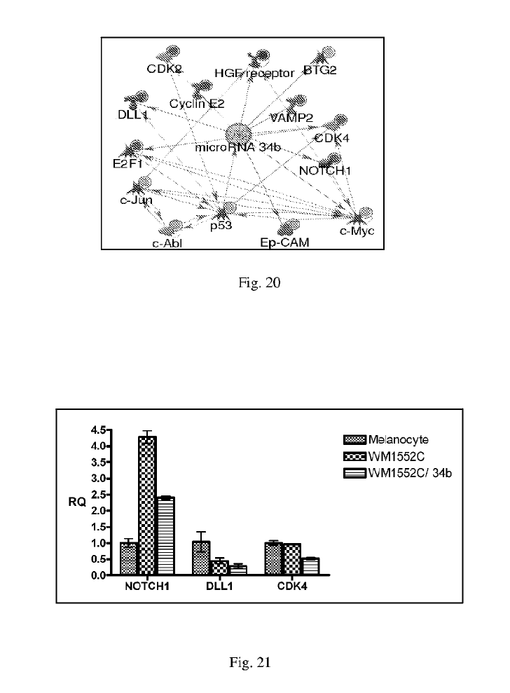

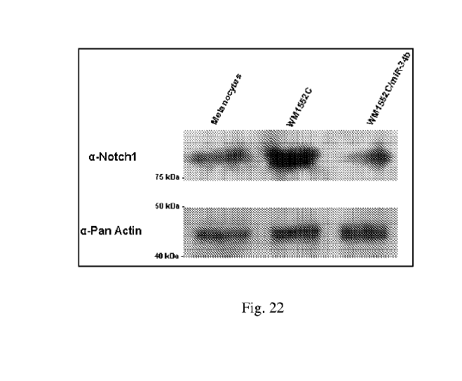

To understand the significance of down-regulation of miR-34b in malignant melanoma, and thus to develop a systematic rationale for its possible future use as a tumor suppressor and biomarker, its mechanism of action or normal cellular function in skin cells must be understood. FIG. 19 serves as an example model. First, candidate target genes of miR-34b were determined using computational resources including miRanda, miRbase, miRNAmap, Tarbase, PicTar, Target ScanS, and DIANA MicroTest (http://www.ncrna.org). Second, first degree interaction maps (FIG. 20) from each putative target gene were generated to computationally evaluate whether the putative target genes were biologically significant. In this preliminary analysis the putative target genes for miR-34b were imported to GeneGo software for further analysis. In FIG. 20, the nodes in the interaction map indicate direct interaction between miR-34b and their putative target genes. From the most connected and representative genes on the network and pathway analyses, it seems that organogenesis, proliferation, development and cell cycle regulation themes are prevalent, along with ECM signaling, ion and/or vesicle transport. Damage response genes can also be observed, and some well-known signaling cascades (MAPK, IP3) were represented among the putative targets of miR-34b. In addition, systems level mapping for target genes of miR-34b also yielded NOTCH1, DLL1, and CDK4 as representative putative targets. Several putative targets including NOTCH1, DLL1, and CDK4 were verified using various procedures such as qRT-PCR and Western blotting. FIG. 21 shows qRT-PCR data confirming that NOTCH 1 expression is down regulated in WM1552C/34b cells (which are cells that express NOTCH1) compared WM1552C which under express miR-34b compared to normal melanocytes. FIG. 22, via Western blotting, confirms that NOTCH1 is up-regulated in WM1552C cells when compared to melanocytes and WM1552C/34b cells. Down-regulation of NOTCH1 in WM1552C/34b compared to WM1552C is due to ectopic expression of miR-34b. In FIG. 22, α-Pan Actin was used as a loading control. ncRNA arrays

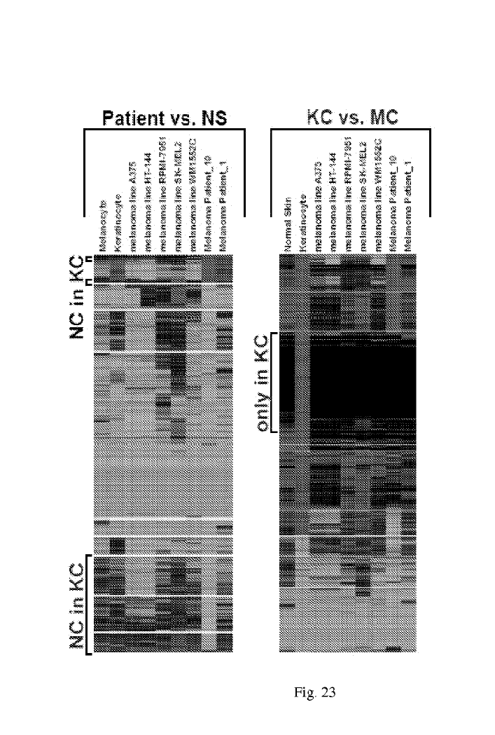

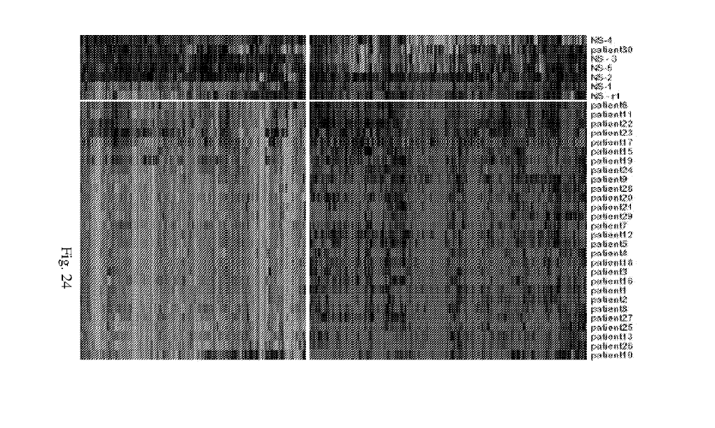

An ncRNA array platform that contains over 10,000 putative (>200 nt) ncRNAs including most of the known ncRNAs in mouse and human was conducted to evaluate ncRNAs within melanoma. Lack of coding potential was estimated by an algorithm that scores various characteristics of protein-coding genes, including open reading frame length, synonymous/non-synonymous base substitution rates and similarity to known proteins. These arrays provide the first generation of tools designed to analyze the dynamic expression of a large subset of ncRNAs in human and mouse and to identify candidate genes for more detailed functional analysis. In addition to the ncRNA content, probes targeting mRNA content from RefSeq are also included, allowing discovery of coordinated expression with associated protein-coding genes. The result of these assays is a list of ncRNA genes (i.e. SEQ ID NO 28-89) and maps of their sequence boundaries. We have profiled total RNA isolated from melanocytes, keratinocytes, normal human skin, melanoma cell lines and melanoma patient samples to identify differentially expressed ncRNAs in cell lines and tissues. ncRNAs expression in all tested samples were compared to either melanocytes or normal skin. We have identified a group of ncRNAs that are specific for (a) melanocytes (b) Keratinocytes (c) Melanoma cell lines (d) Melanoma patients. These results are illustrated in FIG. 23 and FIG. 24 as a hierarchical cluster. Specifically FIG. 23 shows markers which were identified as up-regulated ncRNAs in melanoma cell lines compared to melanocytes and Keratinocytes. Probe DNA sequences for this array include: AK024556 (SEQ ID NO 103) ATTCCAAGGCCTATTAAAATTTCTGAGCATTGCCCATTTCTTTTGCTTTATCTGTAG BC033879 (SEQ ID NO 104) GTTTCCATTGAAGAATATTACGCTCGGGGACAAGCCAGGTGCACGACCAAAAATTTAA AA BC070106 (SEQ ID NO 105) GGTTCTATGTCCCTGCGGCTATGTTTCCAGTGTCCTCTGGGTGTTTCCAAGAGCAACAGG S70348 (SEQ ID NO 106) CAGTCCCTCACCAGTCGCCAGCCTTTCCAAGGTGAGCTTCAGCATTGAGGCCAAGGTGC G In addition, FIG. 24 shows markers that were identified as up-regulated ncRNAs in melanoma patients compared to normal skin. Probe DNA sequences for this array include: AF086032 (SEQ ID NO 107) TTAAATACATGTTTCTAATATTTTAGTGTTTTGTATTTAGGTTATTTATTCTTTGTATCT BC071821 (SEQ ID NO 108) ACGCACTGGGCGTTGAAGCAGTGCTTTCTGGATTAAATACGAAATACTGATGTCACAAG C AK130193 (SEQ ID NO 109) ATTCAAATCTTCATGAATGGCATCACTTGCTTTAGACCCATTTTTTTTTTATTGGTCAGA AL109719 (SEQ ID NO 110) TATTGAATGTACACAAGTAATGATGGAGTTTGATTATATGGTTCATTTTCATTTATCCTA AF085888 (SEQ ID NO 111) CTTAATGTATCTAAGGTTGGCTTAATCCTGTCTAAAATCAAGGTATTGGACTAGAACAA T BC033879 (SEQ ID NO 112) GTTTCCATTGAAGAATATTACGCTCGGGGACAAGCCAGGTGCACGACCAAAAATTTAA AA AK024556 (SEQ ID NO 113) ATTCCAAGGCCTATTAAAATTTCTGAGCATTGCCCATTTCTTTTGCTTTATCTGTAGGAC Probe nomenclature is standard genebank IDs given by NCBI and other organizations.

Furthermore, Tables 4-8 display the results from the ncRNA arrays in which biomarkers were identified as up-regulated ncRNAs in melanoma patients compared to normal skin.

TABLE 4

| SEQ ID NO: | NS 1 | NS 3 | NS 4 | NS 5 | NS 2 | patient 1 | patient 11 |
|---|---|---|---|---|---|---|---|
| SEQ ID NO 38 | −0.18397 | −0.3044 | 1.543579 | 1.197678 | −1.13473 | −0.88439 | −0.85307 |
| SEQ ID NO 39 | 0.663226 | 0.678047 | 0.954746 | 1.862674 | −1.85901 | −0.9977 | −0.56401 |
| SEQ ID NO 28 | −1.48475 | −0.97267 | −1.28892 | −1.13725 | 0.715227 | 2.166854 | 0.436006 |
| SEQ ID NO 40 | 0.406421 | −1.11224 | −0.94685 | −1.17016 | 0.588319 | 2.012531 | 1.525449 |
| SEQ ID NO 29 | 0.601756 | −2.1661 | −2.00071 | −1.85526 | 2.701639 | 3.616065 | 3.759068 |
| SEQ ID NO 41 | 0.035406 | 0.922946 | 2.677648 | 0.993721 | −2.63014 | −2.60073 | −2.70316 |
| SEQ ID NO 42 | 0.923811 | 0.764642 | 1.024359 | −0.26941 | −1.54391 | −1.74569 | −1.99046 |
| SEQ ID NO 43 | −0.96386 | 0.925935 | −0.29176 | 1.300785 | −0.96564 | −1.25726 | −1.20789 |
| SEQ ID NO 44 | −0.48532 | 0.098788 | 0.086925 | 0.715883 | 0.707433 | −1.70419 | −1.50719 |
| SEQ ID NO 45 | 0.484915 | 1.308266 | −0.67635 | −0.00992 | 0.397955 | −1.33311 | −0.55181 |
| SEQ ID NO 46 | −0.06881 | −0.39723 | −0.23184 | −0.09168 | −0.2745 | 1.303026 | −0.02608 |
| SEQ ID NO 47 | −1.87458 | −0.82375 | 1.678205 | −1.65767 | 1.116929 | −2.49453 | −2.24242 |
| SEQ ID NO 48 | −0.46825 | −0.43781 | 1.037048 | 0.250091 | −0.17166 | 1.057506 | −0.35199 |
| SEQ ID NO 49 | −1.86568 | −0.49297 | −0.70937 | −0.64688 | 1.89652 | −2.23469 | −1.65322 |
| SEQ ID NO 50 | −0.06617 | 0.496216 | 2.42918 | −0.10802 | −1.41505 | −2.1832 | −1.8771 |
| SEQ ID NO 51 | 0.608652 | −0.60534 | −1.20891 | 0.901984 | −0.33371 | 2.154459 | 0.410907 |
| SEQ ID NO 52 | −0.26283 | −0.68498 | −0.51959 | −0.7429 | 0.842332 | 1.937199 | 0.848953 |
| SEQ ID NO 53 | −1.49243 | −1.49926 | 5.537652 | 0.223909 | −1.16678 | −2.24401 | −1.61278 |
| SEQ ID NO 54 | −0.24674 | −0.2163 | −0.05091 | 0.24436 | 0.130864 | 1.787402 | −0.31067 |
| SEQ ID NO 55 | −0.44889 | −0.26618 | 2.404464 | 0.680121 | −1.25144 | −1.6333 | −1.3007 |
| SEQ ID NO 56 | −0.6634 | −0.39602 | 1.848257 | 0.39775 | −0.43324 | −1.63561 | −0.80657 |
| SEQ ID NO 30 | 1.312692 | 0.784768 | −2.23409 | 0.605742 | −0.83501 | 1.620619 | 0.365899 |
| SEQ ID NO 31 | 0.505206 | −0.87536 | −2.82416 | −1.28581 | 1.639887 | 3.404738 | 3.91425 |
| SEQ ID NO 32 | 1.412886 | −0.87181 | −1.9883 | −0.82672 | 0.896139 | 0.837592 | 2.486968 |
| SEQ ID NO 57 | −0.66474 | −0.6343 | −0.46891 | 0.197306 | 0.87607 | 1.888573 | 0.419075 |
| SEQ ID NO 33 | 0.314733 | −0.38615 | −0.22077 | −0.44407 | −0.07789 | 0.829768 | 1.344372 |
| SEQ ID NO 58 | 0.811138 | 1.961194 | 1.438036 | −1.79288 | −0.60974 | −1.79635 | −1.448 |
| SEQ ID NO 59 | 0.398442 | 1.874333 | −0.97888 | 0.001484 | −0.55924 | −1.10189 | −1.23864 |
| SEQ ID NO 60 | −1.18088 | −0.1164 | −0.98505 | 0.16024 | 0.573707 | 2.560843 | 2.534506 |
| SEQ ID NO 34 | 0.473513 | −0.20809 | −0.37875 | 0.840526 | −2.09813 | 2.485134 | 2.72187 |
| SEQ ID NO 61 | 0.098344 | −0.01194 | −2.05439 | 1.037346 | 0.365765 | 2.512799 | 0.188703 |
| SEQ ID NO 62 | −0.79163 | −1.64868 | −0.03109 | −0.50345 | 1.357536 | 1.255167 | 0.235902 |
| SEQ ID NO 63 | 0.417787 | 0.65749 | 0.930186 | 1.104023 | −1.6116 | −1.63564 | −0.60608 |
| SEQ ID NO 64 | 1.880259 | −2.20068 | −2.0353 | −1.75378 | 1.544629 | 1.545238 | 1.302229 |
| SEQ ID NO 65 | 0.128576 | 0.056941 | 1.965562 | −0.05983 | −0.86307 | −1.0706 | −0.97498 |
| SEQ ID NO 66 | 0.980446 | −2.66186 | −0.36254 | −1.97365 | 1.647882 | 2.236123 | 1.392401 |
| SEQ ID NO 67 | −0.56844 | 0.461663 | −1.12922 | 0.88806 | −0.60087 | 1.357847 | 1.482893 |
| SEQ ID NO 68 | 1.413835 | 0.729254 | 0.025006 | 0.291339 | −1.23434 | −1.20533 | −0.63355 |
| SEQ ID NO 69 | 0.045796 | 0.211141 | 1.523084 | 0.327199 | −0.76841 | −1.53164 | 0.839082 |
| SEQ ID NO 70 | −0.4794 | 0.547294 | 1.287933 | 0.406162 | −0.3608 | −1.53613 | −1.4013 |
| SEQ ID NO 71 | 0.283534 | −1.3709 | −1.33912 | −1.40043 | 1.838322 | 0.502049 | 0.934465 |
| SEQ ID NO 72 | −0.2695 | −1.43234 | 0.813268 | −1.49026 | 1.988003 | 2.827916 | 4.21377 |
| SEQ ID NO 73 | 0.2452 | −1.2044 | 0.391407 | −0.19911 | 0.074003 | 0.732405 | 2.025477 |
| SEQ ID NO 35 | 1.018082 | −0.78606 | −3.7786 | −1.07429 | 1.860119 | 2.456918 | 2.091677 |
| SEQ ID NO 74 | 0.379389 | −1.17919 | −1.01381 | −0.68072 | 1.22372 | 1.266685 | 0.843177 |
| SEQ ID NO 36 | 1.16162 | −1.68195 | −4.55202 | −1.90393 | 3.170115 | 4.451398 | 3.257058 |
| SEQ ID NO 37 | 0.726121 | −1.27405 | 0.83998 | −1.33197 | −0.6098 | 1.200064 | 0.628988 |
| SEQ ID NO 75 | 0.760053 | −0.28713 | −1.40295 | −1.06098 | −0.14582 | 0.616134 | 1.302725 |
| SEQ ID NO 76 | −0.6465 | 0.78978 | 1.505354 | 0.229184 | −0.44831 | −1.0372 | 0.055859 |
| SEQ ID NO 77 | 0.160148 | 1.103474 | 0.343862 | 1.253049 | −1.52608 | −2.02046 | −1.23412 |
| SEQ ID NO 78 | 1.07479 | 1.595052 | −0.44073 | 3.033057 | −2.93187 | −3.14034 | −2.71638 |
| SEQ ID NO 79 | 0.464777 | 0.192505 | −1.87242 | 1.352979 | −0.60822 | 0.767177 | 1.053219 |
| SEQ ID NO 80 | 0.278782 | 1.102826 | 1.082085 | 0.601858 | −1.61388 | −1.48307 | 0.169525 |
| SEQ ID NO 81 | 0.970403 | 0.746081 | 1.513001 | 1.379423 | −2.66725 | −2.72133 | −2.22553 |
| SEQ ID NO 82 | −2.41961 | −0.8409 | 1.176982 | −1.58337 | 1.269688 | −3.19584 | −3.12161 |
| SEQ ID NO 83 | −1.01222 | −0.27509 | 3.04037 | 0.560441 | −0.88676 | −1.39125 | −1.27496 |
| SEQ ID NO 84 | −0.58759 | −0.45317 | −1.22814 | 1.262172 | −0.48966 | 1.549672 | 1.942534 |
| SEQ ID NO 85 | 0.702044 | 0.343872 | 2.856936 | 1.054756 | −2.74773 | −2.99039 | −2.40689 |
| SEQ ID NO 86 | −0.01792 | 0.012527 | 0.177913 | −0.04539 | −0.08565 | −0.10182 | −0.08185 |
| SEQ ID NO 87 | −1.223 | 3.012369 | 0.555366 | 0.87867 | −1.46553 | −1.52261 | −1.26929 |
| SEQ ID NO 88 | 0.496319 | 0.556697 | 0.906858 | 1.107275 | −1.74257 | −1.72529 | −0.73649 |
| SEQ ID NO 89 | 0.58412 | 0.557906 | 0.782038 | 0.498726 | −1.52403 | −1.32694 | −0.52235 |

TABLE 5

| SEQ ID NO: | patient 12 | patient 13 | patient 15 | patient 16 | patient 17 | patient 18 | patient 19 |
|---|---|---|---|---|---|---|---|
| SEQ ID NO 38 | −0.95345 | −1.02992 | −1.12676 | −0.78905 | −1.02153 | −1.00337 | −0.77286 |
| SEQ ID NO 39 | −1.21328 | −1.1009 | −1.03565 | −1.00566 | −0.70234 | −1.24838 | −0.60061 |
| SEQ ID NO 28 | 2.671165 | 1.062099 | −0.04286 | −0.48722 | 0.32281 | 1.94155 | 0.548629 |
| SEQ ID NO 40 | 0.69372 | 1.373577 | 0.188539 | 0.202627 | −1.13075 | 1.799732 | 0.334211 |
| SEQ ID NO 29 | 2.81875 | 4.531029 | 0.666021 | 2.803833 | 0.19858 | 2.767236 | 0.662528 |
| SEQ ID NO 41 | −2.61743 | −2.58233 | −2.10455 | −2.34843 | −2.5976 | −2.72275 | −0.5732 |
| SEQ ID NO 42 | −1.91945 | −1.36432 | −1.3991 | −1.6423 | −1.14864 | −1.65093 | −2.23111 |
| SEQ ID NO 43 | −1.23116 | −0.96809 | −1.54556 | −1.13262 | −1.54107 | −1.31451 | −1.53066 |
| SEQ ID NO 44 | −1.77933 | −1.85808 | −1.43116 | −1.54665 | −1.86514 | −1.91348 | −1.82998 |
| SEQ ID NO 45 | −1.62112 | −1.56874 | −0.86242 | −1.59835 | −0.00104 | −2.0801 | −0.85835 |
| SEQ ID NO 46 | 0.490796 | 3.436705 | −0.47179 | 0.372759 | −0.23931 | 0.685499 | 2.171824 |
| SEQ ID NO 47 | −2.48926 | −2.49109 | −2.34114 | −2.4863 | −2.21923 | −2.49414 | −2.48549 |
| SEQ ID NO 48 | 0.065979 | 3.093074 | 1.995972 | 3.948065 | 1.359635 | 1.939986 | 3.379954 |
| SEQ ID NO 49 | −1.89901 | −2.4296 | −1.58299 | −2.49967 | −1.27964 | −2.09689 | −2.09439 |
| SEQ ID NO 50 | −2.00018 | −2.13016 | −1.48014 | −1.61278 | −1.74695 | −1.96624 | −0.53471 |
| SEQ ID NO 51 | 0.881063 | 1.291172 | 1.05311 | 0.702135 | 1.599013 | 2.077787 | 0.72279 |
| SEQ ID NO 52 | 1.673065 | 4.366718 | −0.80295 | 1.759657 | 1.125304 | 1.958351 | 0.983528 |
| SEQ ID NO 53 | −2.12195 | −1.89229 | −2.33625 | −1.94784 | −3.09325 | −2.19445 | −2.31855 |
| SEQ ID NO 54 | 0.996951 | 0.847678 | 2.000287 | 1.812308 | 1.68305 | 0.866244 | 3.033667 |
| SEQ ID NO 55 | −1.84294 | −1.70785 | −1.72718 | −1.5595 | −1.22434 | −1.71473 | −1.02924 |
| SEQ ID NO 56 | −1.52824 | −1.58976 | −1.27299 | −1.52975 | −1.14716 | −1.79832 | −1.22795 |
| SEQ ID NO 30 | 1.08744 | 2.246981 | 0.322973 | 2.982383 | 4.196835 | 4.459658 | 0.536288 |
| SEQ ID NO 31 | 1.480632 | 3.61563 | 2.280679 | 3.34256 | 1.262143 | 2.094968 | 2.104682 |
| SEQ ID NO 32 | 0.165665 | 3.047245 | 1.597491 | 1.325765 | 1.559971 | 1.771834 | 2.126226 |
| SEQ ID NO 57 | 1.099001 | 0.167803 | 0.32239 | 0.439754 | 0.675117 | 1.075481 | 0.26276 |
| SEQ ID NO 33 | −0.05783 | 2.215764 | 2.459112 | 2.438238 | 0.248438 | 2.332909 | 1.067887 |
| SEQ ID NO 58 | −1.79175 | −1.58571 | −1.74072 | −1.56898 | −1.1095 | −1.63097 | −1.47052 |
| SEQ ID NO 59 | −1.09389 | −0.94546 | −1.262 | −1.10861 | −0.41373 | −1.23122 | −1.20161 |
| SEQ ID NO 60 | 0.893431 | 1.253727 | 0.820188 | 2.149774 | −0.19269 | 1.892589 | 0.3888 |
| SEQ ID NO 34 | 2.318709 | 1.538448 | 2.073947 | 3.375997 | 2.300359 | 2.792678 | 2.242895 |
| SEQ ID NO 61 | 3.056822 | 2.623271 | 3.280442 | 1.989945 | 1.826711 | 2.8413 | 0.686482 |
| SEQ ID NO 62 | 1.511383 | 1.134827 | 2.797431 | 1.485165 | 0.032091 | 1.1226 | 1.413912 |
| SEQ ID NO 63 | −1.27008 | −1.72788 | −1.13211 | −1.24858 | −0.87712 | −1.31238 | −1.15402 |
| SEQ ID NO 64 | 2.512348 | 1.785465 | 2.763666 | 1.83198 | 1.639232 | 0.955516 | 0.218436 |
| SEQ ID NO 65 | −1.28927 | −1.42346 | −1.38314 | −1.5206 | −0.33299 | −1.40465 | −0.65671 |
| SEQ ID NO 66 | 0.20245 | 3.214596 | 1.223191 | 2.229195 | 0.070371 | 2.44236 | 0.722846 |
| SEQ ID NO 67 | 4.005092 | 1.041516 | 0.960061 | 1.241168 | 6.2781 | 1.041168 | 0.706868 |
| SEQ ID NO 68 | −1.16792 | −1.18105 | −0.97476 | −1.37867 | −0.55655 | −1.33071 | −0.59849 |
| SEQ ID NO 69 | −1.04664 | −1.52484 | −1.05798 | −0.80891 | −1.11157 | −1.09614 | −0.97493 |
| SEQ ID NO 70 | −1.76307 | −1.75204 | −1.31515 | −1.67556 | −0.63737 | −1.78928 | −1.79205 |
| SEQ ID NO 71 | 2.563888 | 0.889395 | 1.971206 | 1.362616 | 1.73855 | 1.735768 | 3.43872 |
| SEQ ID NO 72 | 3.062792 | 2.799667 | 1.047966 | 1.289004 | 0.831137 | 1.40046 | 0.298481 |
| SEQ ID NO 73 | 2.087641 | 1.725928 | 0.153508 | 1.140268 | 1.780365 | 0.459466 | 3.50457 |
| SEQ ID NO 35 | 2.226042 | 3.111243 | 1.769007 | 3.061817 | 4.280921 | 3.66178 | 0.923749 |
| SEQ ID NO 74 | −0.09776 | 2.532365 | 0.120036 | 0.787539 | 2.781802 | 0.95981 | −0.2091 |
| SEQ ID NO 36 | 3.512425 | 4.617795 | 3.087587 | 4.634831 | 1.868099 | 5.754836 | 3.797953 |
| SEQ ID NO 37 | 2.325633 | 1.263248 | 2.288072 | 1.635955 | 1.72629 | 2.225504 | 4.510088 |
| SEQ ID NO 75 | 1.301365 | 1.683382 | 0.539521 | 2.332874 | 0.252565 | 2.064024 | 1.21874 |
| SEQ ID NO 76 | −1.12808 | −1.13435 | −1.12387 | −1.14958 | −0.74232 | −1.29889 | −0.77498 |
| SEQ ID NO 77 | −1.84382 | −2.4965 | −1.33314 | −1.06243 | −1.47072 | −1.56014 | −0.25806 |
| SEQ ID NO 78 | −3.84561 | −3.79686 | −3.31606 | −3.87907 | −3.01162 | −3.96029 | −2.80673 |
| SEQ ID NO 79 | 1.292007 | 1.23109 | 2.142169 | 1.39626 | 1.666731 | 1.371327 | 1.52002 |
| SEQ ID NO 80 | −1.27232 | −1.60329 | −1.1346 | −1.29652 | −0.65053 | −1.49583 | −1.23376 |
| SEQ ID NO 81 | −2.49745 | −2.83069 | −2.46075 | −2.38327 | −1.82043 | −2.62575 | −2.07934 |
| SEQ ID NO 82 | −3.11252 | −3.28004 | −2.84139 | −2.9464 | −3.15007 | −3.09578 | −3.34674 |
| SEQ ID NO 83 | −1.35467 | −1.7009 | −1.22865 | −1.28033 | −0.95491 | −1.04283 | −1.6448 |
| SEQ ID NO 84 | 1.202733 | 1.573044 | 1.193846 | 1.657618 | 1.171341 | 2.221412 | 1.044108 |
| SEQ ID NO 85 | −2.93295 | −3.35298 | −2.56704 | −3.39891 | −1.78054 | −3.17515 | −2.49685 |
| SEQ ID NO 86 | −0.01922 | 1.770661 | 0.927288 | 0.355592 | −0.10907 | 0.34898 | 1.409321 |
| SEQ ID NO 87 | −1.79591 | −1.91369 | −1.60779 | −1.88488 | −0.73443 | −1.89423 | −0.82615 |
| SEQ ID NO 88 | −1.11087 | −1.32302 | −0.92506 | −1.46042 | −0.50644 | −1.47566 | −0.56193 |
| SEQ ID NO 89 | −0.82028 | −1.1709 | −1.0826 | −1.27623 | −0.61702 | −1.2378 | −0.53037 |

TABLE 6

| SEQ ID NO: | patient 19 | patient 2 | patient 20 | patient 21 | patient 22 | patient 23 | patient 24 |
|---|---|---|---|---|---|---|---|
| SEQ ID NO 38 | −0.77286 | −1.1813 | −0.92846 | −1.06758 | −0.75931 | −1.56903 | −1.07787 |
| SEQ ID NO 39 | −0.60061 | −1.04014 | −1.26329 | −1.23237 | −0.30097 | −0.164 | −1.20438 |
| SEQ ID NO 28 | 0.548629 | 2.064209 | 1.334248 | 0.472784 | 1.387633 | 2.286112 | 0.336827 |
| SEQ ID NO 40 | 0.334211 | 1.341171 | 0.999417 | 0.488995 | 0.901056 | 1.124723 | 0.452038 |
| SEQ ID NO 29 | 0.662528 | 3.623402 | 2.377179 | 2.503856 | 1.796304 | 1.689425 | 2.142596 |
| SEQ ID NO 41 | −0.5732 | −2.52904 | −2.54502 | −2.57405 | −2.21237 | −1.1449 | −2.6637 |
| SEQ ID NO 42 | −2.23111 | −1.5031 | −1.77463 | −1.59026 | −1.26027 | −0.10849 | −1.65981 |
| SEQ ID NO 43 | −1.53066 | −1.29995 | −1.23862 | −1.32137 | −1.45119 | −1.44199 | −1.2517 |
| SEQ ID NO 44 | −1.82998 | −1.84275 | −1.65051 | −1.70029 | −1.02906 | −1.95028 | −1.76813 |
| SEQ ID NO 45 | −0.85835 | −1.68788 | −1.79686 | −1.75922 | −1.25036 | 0.22808 | −1.58821 |
| SEQ ID NO 46 | 2.171824 | 0.197957 | 0.394974 | 0.753072 | −0.42748 | 0.608057 | 0.867517 |
| SEQ ID NO 47 | −2.48549 | −2.44204 | −2.49451 | −2.24852 | −2.41044 | −2.40124 | −2.48923 |
| SEQ ID NO 48 | 3.379954 | 0.951453 | 1.712798 | 1.143856 | −0.46806 | −0.45886 | 2.491667 |
| SEQ ID NO 49 | −2.09439 | −3.13084 | −2.12258 | −2.20683 | −2.11766 | −1.78693 | −1.64188 |
| SEQ ID NO 50 | −0.53471 | −2.41547 | −1.77556 | −1.85411 | −1.42728 | −0.15753 | −1.43406 |
| SEQ ID NO 51 | 0.72279 | 0.892244 | 1.113353 | 1.297817 | 0.554273 | 0.191775 | 1.761226 |
| SEQ ID NO 52 | 0.983528 | 2.853243 | −0.57287 | 0.728612 | −0.22771 | −0.70603 | −0.40155 |
| SEQ ID NO 53 | −2.31855 | −2.86702 | −1.90301 | −2.12442 | −2.7031 | −3.17466 | −1.82665 |
| SEQ ID NO 54 | 3.033667 | 0.356974 | 1.876156 | 1.298676 | −0.24656 | 0.763131 | 2.123655 |
| SEQ ID NO 55 | −1.02924 | −1.54037 | −1.81182 | −2.32114 | −1.43923 | −1.27549 | −1.47808 |
| SEQ ID NO 56 | −1.22795 | −2.01394 | −1.69778 | −1.98579 | −1.27665 | −1.27665 | −1.27271 |
| SEQ ID NO 30 | 0.536288 | 4.29971 | 1.366625 | 3.468288 | 1.186881 | 0.307913 | −1.31845 |
| SEQ ID NO 31 | 2.104682 | 2.957517 | 2.853256 | 4.303651 | 2.577691 | 2.860125 | 1.372567 |
| SEQ ID NO 32 | 2.126226 | 1.210694 | 3.195921 | 0.744307 | 0.90223 | 0.861348 | 1.339127 |
| SEQ ID NO 57 | 0.26276 | 1.808397 | 0.192211 | 0.938555 | 1.472936 | 1.122106 | 0.543645 |
| SEQ ID NO 33 | 1.067887 | 0.657529 | 1.404493 | 1.613399 | 0.818509 | −0.40721 | 3.015515 |
| SEQ ID NO 58 | −1.47052 | −1.67536 | −1.13372 | −1.71895 | −1.27998 | −1.75602 | −1.75726 |
| SEQ ID NO 59 | −1.20161 | −0.93392 | −1.13979 | −1.26317 | −0.5383 | −1.16532 | −1.15434 |
| SEQ ID NO 60 | 0.3888 | 2.775617 | 2.118682 | 0.74912 | 3.235841 | 1.416107 | 1.294771 |
| SEQ ID NO 34 | 2.242895 | 2.375226 | 3.044325 | 2.382084 | 2.726615 | 2.642669 | 1.817358 |
| SEQ ID NO 61 | 0.686482 | 1.243506 | −0.03568 | 1.983573 | −0.11431 | 0.552735 | 3.802216 |
| SEQ ID NO 62 | 1.413912 | 1.565419 | 1.836254 | 0.698371 | 0.496815 | −0.5784 | 3.500245 |
| SEQ ID NO 63 | −1.15402 | −1.69405 | −1.23201 | −1.39691 | −0.35615 | −0.04699 | −1.09298 |
| SEQ ID NO 64 | 0.218436 | 2.496045 | 2.310796 | 2.60148 | 1.302407 | 0.991082 | 2.424272 |
| SEQ ID NO 65 | −0.65671 | −1.17661 | −1.28259 | −1.28892 | −1.02999 | −1.59713 | −1.24126 |
| SEQ ID NO 66 | 0.722846 | 2.552707 | 2.462159 | 2.262721 | 1.14959 | 1.370049 | 1.433977 |
| SEQ ID NO 67 | 0.706868 | 2.052929 | 1.071079 | 3.855572 | 1.18628 | 2.369163 | 1.044782 |
| SEQ ID NO 68 | −0.59849 | −1.02912 | −1.37867 | −1.38458 | −0.89534 | −0.71054 | −1.38042 |
| SEQ ID NO 69 | −0.97493 | −1.69682 | −1.42154 | −1.5966 | −0.87825 | −0.21533 | −1.79603 |
| SEQ ID NO 70 | −1.79205 | −1.55287 | −1.61498 | −1.09967 | −1.35125 | −0.78765 | −1.59266 |
| SEQ ID NO 71 | 3.43872 | 0.741301 | 1.577173 | 1.245316 | 1.104884 | 0.257965 | 2.527115 |
| SEQ ID NO 72 | 0.298481 | 2.217068 | 1.011231 | 0.626402 | 0.549926 | 0.091286 | 2.832182 |
| SEQ ID NO 73 | 3.50457 | −0.11725 | 0.630096 | 2.331925 | 0.962202 | 0.07892 | 0.337711 |
| SEQ ID NO 35 | 0.923749 | 1.512872 | 2.507243 | 2.617989 | 0.816656 | 2.728611 | −2.78316 |
| SEQ ID NO 74 | −0.2091 | 2.155304 | 0.877274 | 1.568685 | 0.239834 | 1.77611 | 0.866875 |
| SEQ ID NO 36 | 3.797953 | 3.976906 | 4.668762 | 3.908526 | 3.164022 | 2.960201 | 3.814966 |
| SEQ ID NO 37 | 4.510088 | 1.588798 | 0.993762 | 2.574942 | 0.098798 | 1.771062 | 4.312107 |
| SEQ ID NO 75 | 1.21874 | 0.587491 | 2.944635 | 1.592447 | 1.680653 | 1.545179 | 2.208766 |
| SEQ ID NO 76 | −0.77498 | −1.68152 | −1.18361 | −1.21634 | −0.05441 | 0.732802 | −1.26193 |
| SEQ ID NO 77 | −0.25806 | −2.14106 | −1.52633 | −1.23013 | −0.58924 | 0.229965 | −1.57848 |
| SEQ ID NO 78 | −2.80673 | −3.70003 | −3.81999 | −3.78144 | −1.08887 | −1.0414 | −3.41943 |
| SEQ ID NO 79 | 1.52002 | 0.593787 | 1.494203 | 1.270635 | 1.165021 | 1.44509 | 2.943103 |
| SEQ ID NO 80 | −1.23376 | −1.59354 | −2.05566 | −1.5287 | −0.70731 | −0.11428 | −1.17532 |
| SEQ ID NO 81 | −2.07934 | −2.70435 | −2.52725 | −2.56802 | −1.3874 | −1.28758 | −2.45145 |
| SEQ ID NO 82 | −3.34674 | −3.15288 | −3.11061 | −3.1409 | −2.79067 | −3.009 | −3.01958 |
| SEQ ID NO 83 | −1.6448 | −1.48815 | −1.22813 | −1.44981 | −1.11604 | −1.85472 | −1.06648 |
| SEQ ID NO 84 | 1.044108 | 1.997212 | 1.667663 | 1.56034 | 2.009666 | 2.078309 | 1.532443 |
| SEQ ID NO 85 | −2.49685 | −3.04161 | −3.4136 | −3.39974 | −1.23456 | −0.78402 | −3.13993 |
| SEQ ID NO 86 | 1.409321 | 2.020772 | 0.890605 | 1.055988 | −0.01773 | −0.00853 | 2.719915 |
| SEQ ID NO 87 | −0.82615 | −2.131 | −1.93504 | −2.07877 | −1.31187 | −1.99986 | −3.01105 |
| SEQ ID NO 88 | −0.56193 | −1.50087 | −1.6443 | −1.52675 | −0.80092 | −0.30338 | −1.12765 |
| SEQ ID NO 89 | −0.53037 | −1.08992 | −0.79487 | −1.10436 | −0.91433 | −0.34054 | −0.94091 |

TABLE 7

| SEQ ID NO: | patient 25 | patient 26 | patient 27 | patient 28 | patient 29 | patient 3 | patient 30 |
|---|---|---|---|---|---|---|---|
| SEQ ID NO 38 | −1.36455 | −1.36793 | −1.31724 | −1.18066 | −1.24929 | −1.02019 | 0.332963 |
| SEQ ID NO 39 | −1.66037 | −1.77586 | −1.28675 | −1.44823 | −1.37441 | −0.78521 | 0.023558 |
| SEQ ID NO 28 | 2.417023 | 3.090634 | 2.371103 | 3.315683 | 3.277863 | 2.036528 | −1.35047 |
| SEQ ID NO 40 | 2.169728 | 2.020864 | 1.466993 | 1.363651 | 1.24458 | 1.980291 | 0.734767 |
| SEQ ID NO 29 | 3.283522 | 3.992087 | 2.873002 | 2.810073 | 4.100813 | 2.484602 | −2.06226 |
| SEQ ID NO 41 | −2.4383 | −2.45797 | −2.20419 | −2.5405 | −2.61385 | −2.70244 | 0.710149 |
| SEQ ID NO 42 | −1.5976 | −1.68367 | −1.03774 | −1.33939 | −0.58115 | −1.94101 | 1.332709 |
| SEQ ID NO 43 | −1.54387 | −1.39572 | −1.33866 | −1.38719 | −1.36617 | −1.27205 | −0.14485 |
| SEQ ID NO 44 | −1.97896 | −1.50137 | −1.74405 | −1.67875 | −1.80941 | −1.70524 | 0.981354 |
| SEQ ID NO 45 | −2.09639 | −2.17374 | −1.86017 | −2.06939 | −1.42168 | −1.56043 | 0.879201 |
| SEQ ID NO 46 | 1.427713 | 2.918567 | 1.823524 | 1.252507 | 2.277944 | 0.671777 | 0.445173 |
| SEQ ID NO 47 | −2.45296 | −2.48639 | −2.48855 | −2.48126 | −2.23379 | −2.49427 | −1.52883 |
| SEQ ID NO 48 | 4.69976 | 2.980991 | 4.71182 | 0.210228 | 0.192672 | 3.672375 | −0.33397 |
| SEQ ID NO 49 | −2.47191 | −3.03653 | −2.44677 | −2.62088 | −2.38998 | −1.90086 | −0.92588 |
| SEQ ID NO 50 | −1.80386 | −1.95235 | −1.70723 | −1.73825 | −2.31664 | −1.96228 | −0.33124 |
| SEQ ID NO 51 | 2.552283 | 1.144581 | 1.166454 | 0.878993 | 0.719336 | 2.683112 | −0.43254 |
| SEQ ID NO 52 | 3.264585 | 3.427572 | 0.447899 | 2.064106 | 1.478318 | 1.985905 | −0.58114 |
| SEQ ID NO 53 | −2.86366 | −3.10466 | −2.54167 | −1.90456 | −2.94777 | −2.30611 | −3.04977 |
| SEQ ID NO 54 | 3.286612 | 2.689266 | 4.169981 | 1.501393 | −0.18506 | 2.626619 | −0.11246 |
| SEQ ID NO 55 | −2.10956 | −2.18563 | −2.01929 | −2.04319 | −2.15482 | −1.63831 | −0.50213 |
| SEQ ID NO 56 | −2.40695 | −2.50133 | −1.97031 | −1.95248 | −1.91242 | −1.53454 | −0.55988 |
| SEQ ID NO 30 | 1.31596 | 2.451358 | 4.705932 | 0.803782 | 4.757449 | 3.169211 | 1.116763 |
| SEQ ID NO 31 | 2.408005 | 3.486898 | 2.156462 | 2.249816 | 2.948721 | 2.154271 | −2.88571 |
| SEQ ID NO 32 | 4.071984 | 3.120524 | 3.501185 | 0.996289 | 1.473412 | 0.473654 | −2.04985 |
| SEQ ID NO 57 | 1.558945 | 1.989069 | 1.445573 | 1.064061 | 0.445897 | 2.081128 | −0.53046 |
| SEQ ID NO 33 | 1.628207 | 2.999043 | 3.592583 | 2.239593 | 2.110611 | 0.812325 | −0.28232 |
| SEQ ID NO 58 | −1.52045 | −1.46328 | −1.59498 | −1.70845 | −1.54307 | −1.72271 | −1.63113 |
| SEQ ID NO 59 | −0.9916 | −0.87757 | −1.16125 | −1.23215 | −1.07737 | −1.01954 | −1.04043 |
| SEQ ID NO 60 | 1.225776 | 1.536892 | 1.485321 | 0.69825 | 1.398174 | 2.279241 | 0.029727 |
| SEQ ID NO 34 | 2.013434 | 1.687278 | 2.94521 | 2.485134 | 2.128615 | 2.833394 | −0.12422 |
| SEQ ID NO 61 | 2.349336 | 2.816639 | 1.264154 | 2.584903 | 3.11151 | 1.116562 | 0.253983 |
| SEQ ID NO 62 | 3.363223 | 2.39434 | 1.854942 | 1.417702 | 1.3702 | 1.465539 | −1.54485 |
| SEQ ID NO 63 | −2.09928 | −2.00759 | −1.52539 | −1.60082 | −1.86183 | −1.56509 | 1.621825 |
| SEQ ID NO 64 | 2.501567 | 2.729029 | 2.690369 | 2.931935 | 1.472695 | 1.67302 | −2.09684 |
| SEQ ID NO 65 | −1.38389 | −1.41526 | −1.38825 | −1.42137 | −1.2518 | −1.34372 | 0.580316 |
| SEQ ID NO 66 | 2.534264 | 2.983572 | 2.334058 | 2.748981 | 3.271236 | 2.04372 | −2.55802 |
| SEQ ID NO 67 | 0.362183 | 0.817874 | 1.392177 | 6.658797 | 2.096478 | 1.72109 | −1.19077 |
| SEQ ID NO 68 | −1.60714 | −1.32888 | −1.5151 | −1.55632 | −1.30885 | −0.89902 | −0.10581 |
| SEQ ID NO 69 | −1.17807 | −1.01513 | −0.75943 | −1.69311 | −1.64495 | −1.8068 | 0.845704 |
| SEQ ID NO 70 | −1.47321 | −1.49484 | −1.84789 | −1.54893 | −1.62455 | −1.67292 | −0.66662 |
| SEQ ID NO 71 | 2.03409 | 1.058363 | 1.848554 | 0.926028 | 2.38691 | 0.612649 | −2.46438 |
| SEQ ID NO 72 | 3.663934 | 3.28966 | 2.308319 | 1.04436 | 2.582929 | 2.381888 | −1.3285 |
| SEQ ID NO 73 | 1.570336 | 1.308196 | 0.144742 | 2.165973 | 0.915325 | 1.314058 | −0.26117 |
| SEQ ID NO 35 | 1.773896 | 3.301471 | 3.859837 | 4.108305 | 3.127125 | 3.848335 | −0.83229 |
| SEQ ID NO 74 | 2.598869 | 2.626277 | 0.406194 | 2.421398 | 1.974247 | 1.879418 | −1.07535 |
| SEQ ID NO 36 | 5.08385 | 4.400953 | 4.908616 | 4.877666 | 4.381357 | 3.577209 | −1.69841 |
| SEQ ID NO 37 | 3.470983 | 2.309514 | 0.555399 | 1.731662 | 2.461243 | 0.399338 | −1.17021 |
| SEQ ID NO 75 | −0.30239 | 0.225977 | 3.290686 | 1.546326 | 2.221016 | 0.217715 | −1.4645 |
| SEQ ID NO 76 | −1.54948 | −1.59722 | −1.55452 | −1.59839 | −1.76461 | −1.05102 | 0.406806 |
| SEQ ID NO 77 | −2.21904 | −2.15733 | −1.38695 | −1.16514 | −1.79557 | −1.9159 | 0.644689 |
| SEQ ID NO 78 | −3.51844 | −3.35058 | −3.65295 | −3.21473 | −3.65617 | −3.63364 | 1.597195 |
| SEQ ID NO 79 | 1.205548 | 0.703882 | 0.923715 | 0.585155 | 0.617381 | 1.323552 | 0.514772 |
| SEQ ID NO 80 | −1.99194 | −1.98295 | −1.66471 | −1.4589 | −1.48865 | −1.48682 | 0.129621 |
| SEQ ID NO 81 | −2.88921 | −3.08069 | −2.76998 | −2.66619 | −2.72656 | −2.66378 | 2.229336 |
| SEQ ID NO 82 | −3.42804 | −3.44801 | −3.17971 | −3.34762 | −3.30138 | −2.94252 | −2.48517 |
| SEQ ID NO 83 | −1.42241 | −1.40472 | −1.43001 | −1.28033 | −1.1118 | −1.16396 | −1.72983 |
| SEQ ID NO 84 | 1.328546 | 1.372507 | 1.880005 | 1.062072 | 1.557579 | 1.78879 | 1.104678 |
| SEQ ID NO 85 | −3.19999 | −3.13878 | −3.32756 | −3.26095 | −3.3212 | −2.23632 | 0.634559 |
| SEQ ID NO 86 | 3.621442 | 3.088219 | 5.119098 | −0.07222 | −0.07156 | 1.628209 | 1.028797 |
| SEQ ID NO 87 | −2.32959 | −2.36443 | −2.21372 | −2.16967 | −2.37261 | −1.6373 | 0.79843 |
| SEQ ID NO 88 | −1.93284 | −1.94166 | −2.17288 | −1.86066 | −2.1207 | −1.11767 | 0.720781 |
| SEQ ID NO 89 | −1.67549 | −1.59959 | −0.99561 | −0.96773 | −1.26585 | −1.2821 | 0.759057 |

TABLE 8

| SEQ ID NO: | patient 4 | patient 5 | patient 6 | patient 7 | patient 8 | patient 9 | patient 10 |
|---|---|---|---|---|---|---|---|
| SEQ ID NO 38 | −0.92753 | −1.28828 | −1.61536 | −1.63994 | −1.24378 | −1.18309 | −0.78398 |
| SEQ ID NO 39 | −1.14861 | −1.01662 | −0.94036 | −1.08582 | −0.92822 | −1.30604 | −1.23656 |
| SEQ ID NO 28 | 2.709147 | 1.821876 | −0.13994 | 2.031532 | 1.587939 | 0.792278 | −0.26647 |
| SEQ ID NO 40 | 2.260786 | 0.520009 | 1.497527 | 0.43729 | 1.770502 | 0.462534 | 0.357686 |
| SEQ ID NO 29 | 3.451811 | 2.590454 | 1.011885 | 2.657828 | 3.210596 | 2.109437 | 2.286867 |
| SEQ ID NO 41 | −2.65467 | −2.56554 | −2.67616 | −2.59555 | −2.69901 | −2.45648 | −2.26213 |
| SEQ ID NO 42 | −1.83712 | −1.91554 | −1.44475 | −1.99627 | −2.07274 | −1.56181 | −0.80675 |
| SEQ ID NO 43 | −1.46237 | −1.26942 | −1.48831 | −1.54118 | −1.51115 | −1.22581 | −1.37061 |
| SEQ ID NO 44 | −1.67857 | −1.80682 | −1.07988 | −1.93212 | −2.01945 | −1.86091 | −0.3103 |
| SEQ ID NO 45 | −1.5912 | −1.51868 | −0.75284 | −0.87816 | −2.04168 | −1.8261 | 1.064667 |
| SEQ ID NO 46 | 1.186905 | 0.98534 | 0.043681 | 0.714153 | 1.688494 | 5.175403 | 1.916386 |
| SEQ ID NO 47 | −2.26475 | −2.23379 | −2.07567 | −2.36769 | −1.92706 | −2.48363 | −1.68505 |
| SEQ ID NO 48 | 3.353043 | 0.236199 | −0.1733 | 0.648005 | 3.308853 | −0.27592 | 2.286464 |
| SEQ ID NO 49 | −2.3226 | −1.9036 | 0.798566 | −1.47703 | −1.97443 | −2.21394 | −4.42945 |
| SEQ ID NO 50 | −2.10154 | −1.78158 | −1.15882 | −1.97799 | −1.67992 | −2.02985 | −1.53708 |
| SEQ ID NO 51 | 2.263838 | 1.041894 | 1.115276 | 1.140498 | 2.047594 | 0.778933 | 0.857231 |
| SEQ ID NO 52 | 3.66346 | 1.551986 | 0.78088 | 0.97789 | 2.426471 | 0.867841 | 0.733244 |
| SEQ ID NO 53 | −2.08008 | −2.01956 | −1.88159 | −2.06815 | −1.87849 | −2.19458 | −3.16006 |
| SEQ ID NO 54 | 3.178791 | 0.254665 | 0.567959 | 1.299779 | 1.14429 | −0.14005 | 0.436346 |
| SEQ ID NO 55 | −1.74672 | −1.56474 | −1.13451 | −1.9789 | −1.06535 | −2.02941 | −1.19066 |
| SEQ ID NO 56 | −1.64021 | −1.4872 | −1.12921 | −1.28187 | −1.31401 | −1.81753 | −2.92197 |
| SEQ ID NO 30 | 2.472328 | 3.839941 | 2.19069 | 4.638524 | 2.888008 | 0.450827 | 3.549002 |
| SEQ ID NO 31 | 2.238236 | 2.367913 | 1.428963 | 4.220045 | −0.41778 | 2.587519 | 3.165212 |
| SEQ ID NO 32 | 2.716652 | 1.509845 | 0.743903 | 1.567785 | 2.60605 | 1.062046 | 0.728609 |
| SEQ ID NO 57 | 1.612724 | 2.210788 | 1.679967 | 0.60993 | 1.985396 | 0.645023 | 0.502274 |
| SEQ ID NO 33 | 3.512035 | 0.699779 | 1.767996 | 2.378708 | 2.278138 | 0.313573 | 1.984065 |
| SEQ ID NO 58 | −0.43247 | −1.70682 | −1.80234 | −1.2619 | −1.82519 | −1.56856 | −1.22274 |
| SEQ ID NO 59 | −1.16837 | −1.25461 | −1.21165 | −0.93251 | −0.99368 | −1.25805 | −0.96878 |
| SEQ ID NO 60 | 2.327148 | 0.865485 | −1.21782 | 2.958007 | 0.315417 | 1.738715 | 2.002092 |
| SEQ ID NO 34 | 2.701105 | 2.72187 | 2.606437 | 3.005041 | 2.353917 | 1.958101 | −1.78707 |
| SEQ ID NO 61 | 1.120103 | 3.91709 | 0.99202 | 2.069296 | 1.860453 | 1.694394 | 3.678313 |
| SEQ ID NO 62 | 3.469573 | 1.994171 | 0.575088 | 2.15236 | 0.260901 | 0.580093 | 1.263919 |
| SEQ ID NO 63 | −1.39559 | −1.31495 | −0.59211 | −1.30476 | −1.25966 | −1.42912 | −1.41696 |
| SEQ ID NO 64 | 4.824378 | 1.545613 | 1.799599 | 1.388793 | 2.2638 | 1.339119 | 2.481019 |
| SEQ ID NO 65 | −1.21536 | −1.30095 | −1.64345 | −1.26403 | −1.39109 | −1.26673 | −0.30457 |
| SEQ ID NO 66 | 2.799394 | 1.982666 | 2.127441 | 2.319419 | 2.869384 | 1.497646 | 1.586674 |
| SEQ ID NO 67 | 0.653214 | 4.146274 | 1.480872 | 2.486415 | 1.375215 | 1.110999 | 3.887087 |
| SEQ ID NO 68 | −1.23346 | −1.82841 | −0.64413 | −2.07801 | −0.80967 | −1.12146 | −1.44478 |
| SEQ ID NO 69 | −1.36938 | −1.79732 | −0.55261 | −1.8141 | −1.77721 | −1.61799 | −1.69559 |
| SEQ ID NO 70 | −1.21676 | −1.52057 | −1.48279 | −1.75132 | −2.18222 | −1.36622 | −0.69309 |
| SEQ ID NO 71 | 1.335923 | 2.628361 | 1.002314 | 1.935892 | 0.722133 | 0.159163 | 1.786534 |
| SEQ ID NO 72 | 2.565767 | 1.096361 | 2.335584 | 2.665174 | 3.322852 | −0.66294 | 2.244665 |
| SEQ ID NO 73 | 2.476865 | −0.06181 | 1.506446 | −0.17903 | 1.517797 | 1.006572 | 1.767291 |
| SEQ ID NO 35 | 3.278374 | 2.520887 | 2.070299 | 3.025305 | 2.140174 | −0.19312 | 3.606105 |
| SEQ ID NO 74 | 2.326328 | 0.323873 | 0.8281 | 2.015796 | 1.308772 | 0.615668 | 2.172137 |
| SEQ ID NO 36 | 4.180785 | 4.049742 | 3.519736 | 4.600501 | 4.360499 | 3.565296 | 2.81807 |
| SEQ ID NO 37 | 0.777341 | 2.424721 | −0.34943 | 1.932962 | 1.30241 | 2.618539 | 3.899319 |
| SEQ ID NO 75 | 1.06249 | 1.072137 | 1.853037 | 2.113925 | 2.604759 | 1.117018 | 0.696754 |
| SEQ ID NO 76 | −1.29675 | −1.03592 | 0.39533 | −1.74103 | −1.68072 | −1.28403 | −0.61694 |
| SEQ ID NO 77 | −2.04189 | −1.54905 | −0.83828 | −1.63894 | −1.64426 | −2.05347 | −2.14564 |
| SEQ ID NO 78 | −3.59298 | −3.40956 | −1.8308 | −3.86136 | −3.60731 | −3.9856 | −3.49614 |
| SEQ ID NO 79 | 1.428227 | 1.429766 | 1.201296 | 1.729068 | 1.727739 | 1.129133 | 0.180842 |
| SEQ ID NO 80 | −1.4489 | −1.20751 | −0.98203 | −1.21327 | −1.33774 | −1.38329 | −1.81549 |
| SEQ ID NO 81 | −2.73994 | −2.51546 | −1.77562 | −2.53264 | −2.25039 | −2.57941 | −3.02662 |
| SEQ ID NO 82 | −3.11035 | −2.73314 | −1.17912 | −3.13339 | −2.95453 | −3.05637 | −3.4499 |
| SEQ ID NO 83 | −1.69088 | −1.15569 | −0.6047 | −0.94958 | −0.95335 | −1.01611 | −1.38122 |
| SEQ ID NO 84 | 1.045907 | 0.99586 | 0.37409 | 2.004605 | 1.103943 | 1.549672 | 0.256062 |
| SEQ ID NO 85 | −3.35251 | −3.14378 | −1.87817 | −3.33069 | −3.24115 | −3.14962 | −2.87956 |
| SEQ ID NO 86 | 5.075849 | −0.09781 | −0.05485 | −0.10787 | 0.750996 | 0.024006 | 1.096999 |
| SEQ ID NO 87 | −2.99942 | −2.97162 | −2.3216 | −2.46776 | −2.76025 | −1.53566 | −1.88128 |
| SEQ ID NO 88 | −1.15771 | −1.19155 | −0.882 | −1.16539 | −1.20601 | −1.55067 | −1.88257 |
| SEQ ID NO 89 | −1.43365 | −1.04904 | −0.65354 | −0.51616 | −1.13656 | −1.44178 | −1.64281 |

When normal skin expression was compared to all other samples, a group of up-regulated ncRNAs were identified in melanoma patients' as well as in keratinocytes. This indicates that patients' samples contain significant amount of keratinocytes. Keratinocytes specific signatures were subtracted and resultant ncRNAs were validated by qRT-PCR and Northern blots. Results point out the presence of a group of melanoma specific ncRNAs (i.e. SEQ ID NO 28-89).

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

1. A method of evaluating the risk or progression of melanoma in a subject comprising quantifying the amount of at least one biomarker present in a biological sample derived from the subject, wherein the biomarker comprises an ncRNA.

2. (canceled)

3. The method of claim 1, wherein the ncRNA is an miRNA.

4. The method of claim 1, wherein the biomarker comprising the ncRNA comprises SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-16), SEQ ID NO 7 (miR-let-7a), SEQ ID NO 8 (miR-let-7b), SEQ ID NO 9 (miR-let-7c), SEQ ID NO 10 (miR-let-7d), SEQ ID NO 11 (miR-let-7e), SEQ ID NO 12 (miR-let-7f), SEQ ID NO 13 (miR-let-7g), SEQ ID NO 14 (miR-let-7i), SEQ ID NO 15 (miR-125a), SEQ ID NO 16 (miR-125b), SEQ ID NO 17 (miR-15b), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof.

5. The method of claim 1, further comprising comparing the amount of the at least one biomarker in a control biological sample derived from a subject not having melanoma to identify an increased risk from melanoma.

6. The method of claim 1, wherein identifying an increased risk or progression of melanoma comprises determining that the amount of the at least one biomarker in the biological sample is significantly higher than the control concentration of the at least one biomarker in a control biological sample, and wherein the at least one biomarker comprises SEQ ID NO 6 (miR-16), SEQ ID NO 7 (miR-let-7a), SEQ ID NO 8 (miR-let-7b), SEQ ID NO 9 (miR-let-7c), SEQ ID NO 10 (miR-let-7d), SEQ ID NO 11 (miR-let-7e), SEQ ID NO 12 (miR-let-7f), SEQ ID NO 13 (miR-let-7g), SEQ ID NO 14 (miR-let-7i), SEQ ID NO 15 (miR-125a), SEQ ID NO 16 (miR-125b), SEQ ID NO 17 (miR-15b), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 40, SEQ ID NO 46, SEQ ID NO 48, SEQ ID NO 51, SEQ ID NO 52, SEQ ID NO 54, SEQ ID NO 57, SEQ ID NO 60, SEQ ID NO 61, SEQ ID NO 62, SEQ ID NO 64, SEQ ID NO 66, SEQ ID NO 67, SEQ ID NO 71, SEQ ID NO 72, SEQ ID NO 73, SEQ ID NO 74, SEQ ID NO 75, SEQ ID NO 79, SEQ ID NO 84, SEQ ID NO 86, or any combination thereof.

7. The method of claim 1, wherein identifying an increased risk or progression of melanoma comprises determining that the amount of the at least one biomarker in a biological sample is significantly lower than the control concentration of the at least one biomarker in a control biological sample, and wherein the at least one biomarker comprises SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 38, SEQ ID NO 39, SEQ ID NO 41, SEQ ID NO 42, SEQ ID NO 43, SEQ ID NO 44, SEQ ID NO 45, SEQ ID NO 47, SEQ ID NO 49, SEQ ID NO 50, SEQ ID NO 53, SEQ ID NO 55, SEQ ID NO 56, SEQ ID NO 58, SEQ ID NO 59, SEQ ID NO 63, SEQ ID NO 65, SEQ ID NO 68, SEQ ID NO 69, SEQ ID NO 70, SEQ ID NO 76, SEQ ID NO 77, SEQ ID NO 78, SEQ ID NO 80, SEQ ID NO 81, SEQ ID NO 82, SEQ ID NO 83, SEQ ID NO 85, SEQ ID NO 87, SEQ ID NO 88, SEQ ID NO 89, or any combination thereof.

8. The method of claim 1, wherein the method comprises quantifying the amount of at least two ncRNAs having the sequence SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-let-7a), SEQ ID NO 7 (miR-let-7b), SEQ ID NO 8 (miR-let-7c), SEQ ID NO 9 (miR-let-7d), SEQ ID NO 10 (miR-let-7e), SEQ ID NO 11 (miR-let-7f), SEQ ID NO 12 (miR-let-7g), SEQ ID NO 13 (miR-let-7i), SEQ ID NO 14 (miR-125a), SEQ ID NO 15 (miR-125b), SEQ ID NO 16 (miR-15b), SEQ ID NO 17 (miR-16), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof.

9. The method of claim 1, wherein the method comprises quantifying the amount of at least three ncRNAs having the sequence SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-let-7a), SEQ ID NO 7 (miR-let-7b), SEQ ID NO 8 (miR-let-7c), SEQ ID NO 9 (miR-let-7d), SEQ ID NO 10 (miR-let-7e), SEQ ID NO 11 (miR-let-7f), SEQ ID NO 12 (miR-let-7g), SEQ ID NO 13 (miR-let-7i), SEQ ID NO 14 (miR-125a), SEQ ID NO 15 (miR-125b), SEQ ID NO 16 (miR-15b), SEQ ID NO 17 (miR-16), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof.

10. The method of claim 1, wherein the at least one biomarker comprises at least four ncRNAs having the sequence SEQ ID NO 1 (miR-211), SEQ ID NO 2 (miR-34b), SEQ ID NO 3 (miR-375), SEQ ID NO 4 (miR-204), SEQ ID NO 5 (miR-99a), SEQ ID NO 6 (miR-let-7a), SEQ ID NO 7 (miR-let-7b), SEQ ID NO 8 (miR-let-7c), SEQ ID NO 9 (miR-let-7d), SEQ ID NO 10 (miR-let-7e), SEQ ID NO 11 (miR-let-7f), SEQ ID NO 12 (miR-let-7g), SEQ ID NO 13 (miR-let-7i), SEQ ID NO 14 (miR-125a), SEQ ID NO 15 (miR-125b), SEQ ID NO 16 (miR-15b), SEQ ID NO 17 (miR-16), SEQ ID NO 18 (miR-199a), SEQ ID NO 19 (miR-21), SEQ ID NO 20 (miR-214), SEQ ID NO 21 (miR-221), SEQ ID NO 22 (miR-222), SEQ ID NO 23 (miR-23a), SEQ ID NO 24 (miR-23b), SEQ ID NO 25 (miR-26a), SEQ ID NO 26 (miR-30c), SEQ ID NO 27 (miR-320), SEQ ID NO 28, SEQ ID NO 29, SEQ ID NO 30, SEQ ID NO 31, SEQ ID NO 32, SEQ ID NO 33, SEQ ID NO 34, SEQ ID NO 35, SEQ ID NO 36, SEQ ID NO 37, SEQ ID NO 38, SEQ ID NO 39, SEQ ID NO 40, SEQ ID NO 41, SEQ ID NO 42, SEQ ID NO 43, SEQ ID NO 44, SEQ ID NO 45, SEQ ID NO 46, SEQ ID NO 47, SEQ ID NO 48, SEQ ID NO 49, SEQ ID NO 50, SEQ ID NO 51, SEQ ID NO 52, SEQ ID NO 53, SEQ ID NO 54, SEQ ID NO 55, SEQ ID NO 56, SEQ ID NO 57, SEQ ID NO 58, SEQ ID NO 59, SEQ ID NO 60, SEQ ID NO 61, SEQ ID NO 62, SEQ ID NO 63, SEQ ID NO 64, SEQ ID NO 65, SEQ ID NO 66, SEQ ID NO 67, SEQ ID NO 68, SEQ ID NO 69, SEQ ID NO 70, SEQ ID NO 71, SEQ ID NO 72, SEQ ID NO 73, SEQ ID NO 74, SEQ ID NO 75, SEQ ID NO 76, SEQ ID NO 77, SEQ ID NO 78, SEQ ID NO 79, SEQ ID NO 80, SEQ ID NO 81, SEQ ID NO 82, SEQ ID NO 83, SEQ ID NO 84, SEQ ID NO 85, SEQ ID NO 86, SEQ ID NO 87, SEQ ID NO 88, SEQ ID NO 89, or any combination thereof.

11. The method of claim 1, wherein the biomarker comprises SEQ ID NO 1 (miR-211).

12. The method of claim 1, wherein the biomarker comprises SEQ ID NO 2 (miR-34b).

13. The method of claim 1, wherein the biomarker comprises SEQ ID NO 3 (miR-375)

14. The method of claim 1, wherein the biomarker comprises SEQ ID NO 1 (miR-211) and SEQ ID NO 2 (miR-34b).

15. The method of claim 1, wherein the biomarker comprises SEQ ID NO 1 (miR-211) and SEQ ID NO 3 (miR-375).

16. The method of claim 1, wherein the biomarker comprises SEQ ID NO 2 (miR-34b) and SEQ ID NO 3 (miR-375).

17. The method of claim 1, wherein the biomarker comprises SEQ ID NO 1 (miR-211), SEQ ID NO 2 (miR-34b), SEQ ID NO 3 (miR-375), or any combination thereof.

18. The method of claim 1, wherein the biological sample comprises a biopsy, a skin biopsy, a mole or nevus biopsy, blood, serum, cultured cells, or any combination thereof.

19. The method of claim 1, wherein the quantifying step comprises an ncRNA microarray analysis, RT-PCR, qRT-PCR, northern blotting, western blotting, DNA sequencing, or any combination thereof.
